(19) United States
(12) Patent Application Publication
(10) Pub. No.: US 2023/0134677 A1
(43) Pub. Date: May 4, 2023

(54) ANTISENSE OLIGONUCLEOTIDES FOR USE IN THE TREATMENT OF USHER SYNDROME

(71) Applicant: ProQR Therapeutics II B.V., Leiden (NL)

(72) Inventors: Jim Swildens, Leiden (NL); Saskia Jacoba Petronella Haast, Leiden (NL)

(21) Appl. No.: 17/908,199

(22) PCT Filed: Mar. 3, 2021
(86) PCT No.: PCT/EP2021/055292

§ 371 Date: Aug. 30, 2022

(30) Foreign Application Priority Data
Mar. 4, 2020 (EP) 20160886.6
Jun. 5, 2020 (EP) 20178470.9

(51) Int. Cl. C12N 15/113; C12N 15/11

(52) U.S. Cl. CPC C12N 15/1138; C12N 15/111; C12N 2310/11; C12N 2310/315; C12N 2310/321; C12N 2320/33

(57) ABSTRACT

The invention relates to the fields of medicine and to antisense oligonucleotides (AONs) that are capable of skipping exon 50 from human USH2A pre-mRNA and that may be used in the treatment, prevention and/or delay of Usher syndrome type II and/or USH2A-associated non syndromic retina degeneration.

### FIELD OF THE INVENTION

The invention relates to the field of medicine. It relates to single-stranded antisense oligonucleotides (AONs) for use in the treatment, prevention and/or delay of eye diseases, preferably Usher syndrome, and/or USH2A-associated retinal degeneration.

### BACKGROUND OF THE INVENTION

Usher syndrome (USH, or just ‘Usher’) and non-syndromic retinitis pigmentosa (NSRP) are degenerative diseases of the retina. Usher is clinically and genetically heterogeneous and by far the most common type of inherited deaf-blindness in man (1 in 6,000 individuals; Kimberling et al. 2010. Genet Med 12:512-516). The hearing impairment in Usher patients is mostly stable and congenital and can be partly compensated by hearing aids or cochlear implants. The degeneration of photoreceptor cells in Usher and NSRP is progressive and often leads to complete blindness between the third and fourth decade of life, thereby leaving time for therapeutic intervention. Mutations in the USH2A gene are the most frequent cause of Usher syndrome type IIa explaining up to 50% of all Usher patients worldwide and as indicated by McGee et al. (2010. J Med Genet 47(7):499-506), also the most prevalent cause of NSRP in the USA, likely accounting for 12-25% of all cases of retinitis pigmentosa (RP). The mutations are spread throughout the 72 USH2A exons and their flanking intron sequences, and consist of nonsense and missense mutations, deletions, duplications, large rearrangements, and splicing variants. Exon 13 is by far the most frequently mutated exon with two founder mutations (c.2299delG (p.E767SfsX21) in USH2 patients and c.2276G>T (p.C759F) in NSRP patients). A deep-intronic mutation in intron 40 of USH2A (c.7595-2144A>G) creates a cryptic high-quality splice donor site in intron 40 resulting in the inclusion of an aberrant exon of 152 bp (Pseudo Exon 40, or PE40) in the mutant USH2A mRNA, that causes premature termination of translation (Vache et al. 2012. Human Mutation 33(1):104-108). For exon 50, fourteen pathogenic mutations have been reported, with an estimated 400 patients suffering from one or more of these mutations in the western world. Examples of these pathogenic mutations are c.9799T>C, c.9958+69C>T, c.9815C>T, c.9811delA, and c.9958G>T.

Usher and other retinal dystrophies have for long been considered as incurable disorders. Several phase I/II clinical trials using gene augmentation therapy have led to promising results in selected groups of LCA/RP/USH patients with mutations in the RPE65 gene (Bainbridge et al. 2008. N Engl J Med 358, 2231-2239) and MYO7A gene (Hashimoto et al. 2007. Gene Ther 14(7):584-594). The size of the coding sequence (15,606 bp) and alternative splicing of the USH2A gene and mRNA hamper gene augmentation therapy due to the currently limiting cargo size of many available vectors (such as adeno-associated virus (AAV) and lentiviral vectors).

Over the last decade several antisense oligonucleotide (AON)-based therapies for the eye have been developed (WO2012/168435; WO2013/036105; WO2015/004133; WO2016/005514; WO2016/034680; WO2016/135334; WO2017/060317; WO2017/186739; WO2018/055134; WO2018/189376), with a mutated CEP290-targeting AON (sepofarsen for Leber's Congenital Amaurosis type 10, or LCA10) proceeding into clinical trials showing very promising effects. AONs are small polynucleotide molecules (generally 16- to 25-mers) that may modulate splicing as their sequence is complementary to that of target pre-mRNA molecules. The envisioned mechanism is such that upon binding of an AON to a target sequence, with which it is complementary, the targeted region within the pre-mRNA is no longer available for splicing factors which in turn results in skipping of the targeted exon. Therapeutically, this methodology can be used in two ways: a) to redirect normal splicing of genes in which mutations activate cryptic splice sites and b) to skip exons that carry mutations such that the reading frame of the mRNA remains intact and a (partially or fully) functional protein is made. For the USH2A gene, 28 exons can potentially be skipped without disturbing the overall reading frame of the transcript. These in-frame exons include exon 13 and exon 50. WO2016/005514 discloses exon skipping AONs for the USH2A pre-mRNA, directed at skipping of exon 13, exon 50 and PE40. WO2017/186739 discloses additional and improved PE40 skipping AONs and WO2018/055134 discloses additional and improved exon 13 skipping AONs.

There is a need for additional, alternative and potentially more effective AONs that would modulate splicing events elsewhere in the USH2A pre-mRNA and cause the skip of other (or the same) in-frame exons, while then restoring (at least partially) the function of usherin, which is the protein encoded by the USH2A gene.

As mentioned above, exon 50 in the human USH2A gene is of interest because of the multiple mutations that have been identified in this exon and skipping exon 50 does not result in a frame-shift. It is an objective of the present invention to provide improved AONs that can be used in a convenient therapeutic strategy for the prevention, treatment or delay of Usher and/or NSRP caused by mutations in exon 50 of the human USH2A gene.

### SUMMARY OF THE INVENTION

The present invention relates to an antisense oligonucleotide (AON) capable of skipping exon 50 from human USH2A pre-mRNA, wherein the AON comprises a 14 to 22 consecutive nucleotide sequence that is 90% to 100% complementary, preferably 100% complementary to a consecutive sequence within SEQ ID NO:53. Preferably, the AON according to the invention consists of 16, 17, 18, 19, 20, 21 or 22 nucleotides. In one preferred aspect, the AON according to the invention consists of a sequence selected from the group consisting of SEQ ID NO:48, 15, 16, 45, 46, 47, 49, 50, 56 to 100, 54, 55, and 101 to 106 (herein referred to as AON48, AON15, AON16, AON45, AON46, AON47, AON49, AON50, and AON53 to AON105, respectively, see FIG. 1).

Preferably, the AON according to the invention consists of a sequence selected from the group consisting of SEQ ID NO: 58, 59, 104, 48, 57, 56, 15, 73, 50, 81, 101, 102, 72, 68, 71 and 66 (herein referred to as AON55, AON56, AON103, AON48, AON54, AON53, AON15, AON70, AON50, AON78, AON100, AON101, AON69, AON65, AON68, and AON63 respectively). In a preferred embodiment, the AON of the present invention is an oligoribonucleotide, and in a more preferred embodiment, the AON according to the invention comprises at least one 2′-O-methoxyethyl (2′-MOE) or at least one 2′-O-methyl (2′-OMe) modification. In a particularly preferred aspect, all nucleotides of the AON according to the invention are 2′-MOE modified or all nucleotides of the AON according to the invention are 2′-OMe modified. In yet another preferred embodiment, the AON according to the invention comprises at least one non-naturally occurring internucleosidic linkage, such as a phosphorothioate (PS) linkage, more preferably, wherein all sequential nucleosides are interconnected by PS linkages.

In another embodiment, the invention relates to a vector, preferably a viral vector, expressing an AON according to the invention. In another embodiment, the invention relates to a pharmaceutical composition comprising an AON according to the invention, or a (viral) vector according to the invention, and a pharmaceutically acceptable carrier.

In another embodiment, the invention relates to an AON according to the invention, a (viral) vector according to the invention, or a pharmaceutical composition according to the invention for use in the treatment, prevention or delay of an USH2A-related disease or a condition requiring modulating splicing of USH2A pre-mRNA, such as Usher syndrome type II.

The invention also relates to a use of an AON according to the invention, a (viral) vector according to the invention, or a pharmaceutical composition according to the invention for the preparation of a medicament for the treatment, prevention or delay of an USH2A-related disease or a condition requiring modulating splicing of USH2A pre-mRNA, such as Usher syndrome type II.

The invention furthermore relates to a method for the treatment of a USH2A-related disease or condition requiring modulating splicing of USH2A pre-mRNA of an individual in need thereof, said method comprising contacting a cell of said individual with an AON according to the invention, a (viral) vector according to the invention, or a pharmaceutical composition according to the invention.

### BRIEF DESCRIPTION OF THE DRAWINGS

FIGS. 1A and B show the 5′ to 3′ DNA sequence of human USH2A exon 50 (in bold, upper case; SEQ ID NO:52) preceded by the 3′ part of intron 49 (lower case) and followed by the 5′ part of intron 50 (lower case). The intron sequences together with the exon sequences are provided as SEQ ID NO:51. Represented here is the DNA sequence, although the target sequence of the AONs according to the invention is its corresponding pre-mRNA sequence. Below the target sequence the sequences are given of the antisense oligonucleotides outlined herein (3′ to 5′ with their respective numbering AON 1 to AON50 and AON53 to AON 105). Two AON sequences are in italic font: Radboud-1/AON20 and Radboud-2/AON21 that are known from WO 2016/005514. Underlined are the most 3′ terminal 22 nucleotides of exon 50 together with the most 5′ terminal 5 nucleotides of intron 50, together forming the 27 nucleotide target sequence (SEQ ID NO:53) that is found to be of particular interest as shown in the accompanying examples.

FIG. 2 shows the percentage of exon 50 skip after ddPCR using a transfection of 2′-MOE modified oligonucleotides AON1 to AON21 on WERI-Rb1 cells. The three negative controls were no transfection (NT), an unrelated negative control oligonucleotide (NC), and a reverse transcriptase (RT) control. The bars below the diagram represents intron 49, exon 50 and intron 50 of the human USH2A pre-mRNA and the order of the AONs in this diagram is indicative of the position of their target sequence in this pre-mRNA (see FIG. 1).

FIG. 3 shows the percentage of exon 50 skip after ddPCR using a transfection of 2′-MOE modified oligonucleotides as depicted. The negative controls were as given in FIG. 2, with the addition of a mock transfection. The order of AONs given on the x-axis represents the distribution of their respective target sequences in exon 50 of human USH2A pre-mRNA.

FIG. 4 shows the percentage of exon 50 skip after ddPCR using a transfection of 2′-MOE modified oligonucleotides as depicted. The negative controls were a mock transfection and a negative control oligonucleotide. The order of AONs given on the x-axis represents the distribution of their respective target sequences in exon 50 of human USH2A pre-mRNA.

FIG. 5 shows the difference in exon skipping percentages observed after transfection (A) and gymnotic uptake (B) in WERI-Rb1 cells of the AONs as depicted.

FIG. 6 shows the percentage of exon 50 skip observed after gymnotic uptake of the AONs depicted at the bottom, again in WERI-RB1 cells. NC is a negative control oligonucleotide; NT is non-treated sample and RT is a reverse transcriptase control (no cDNA).

FIG. 7 shows the percentage of exon 50 skip in USH2A RNA observed in human eye-cups (organoids) after treatment with AON3, AON21, AON43, AON48, AON55, AON101, AON103, and a negative control oligonucleotide.

FIG. 8 shows the percentage of exon 50 skip in USDH2A RNA using two different concentrations of AON3, AON21, AON43, AON48, AON55, AON101, AON103 (1.5 μM and 7.5 μM) in human eye-cups (organoids).

### DETAILED DESCRIPTION

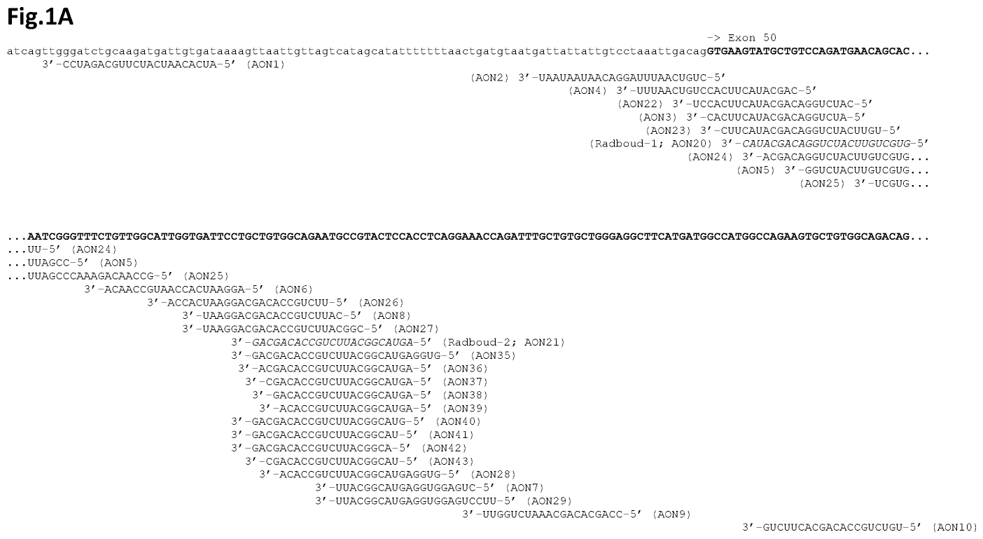

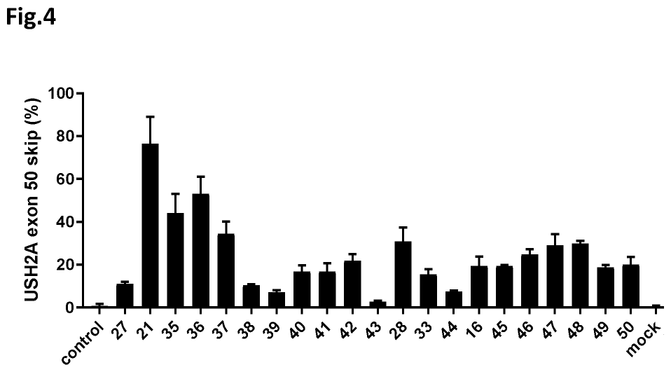

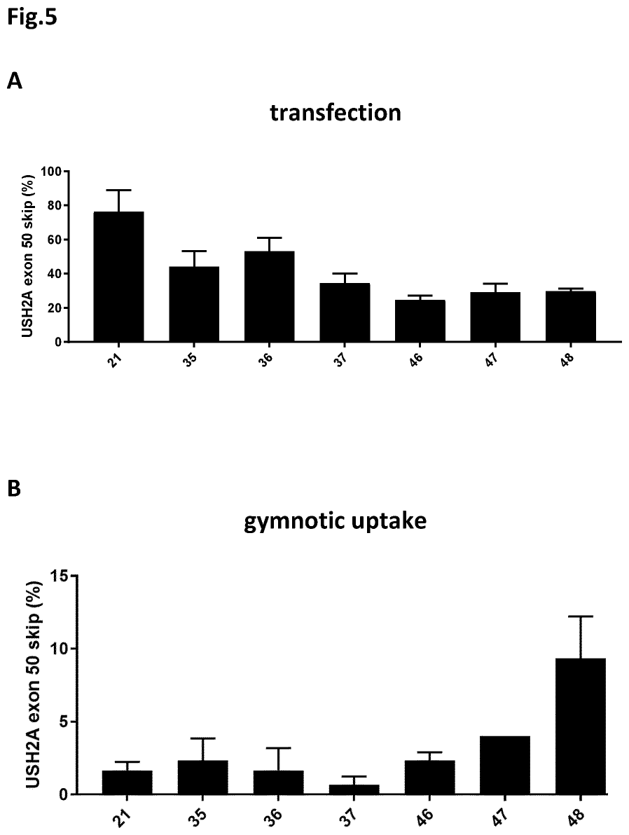

The present invention relates to specific antisense oligonucleotides (AONs) that can block the inclusion of exon 50 mRNA sequence in the mRNA coding for human usherin protein. More specifically, the present invention relates to an AON for skipping exon 50 in human USH2A pre-mRNA, wherein the AON under physiological conditions binds to and/or is complementary to the most 3′ located nucleotides of exon 50 of human USH2A. Preferably, the AON of the present invention is no longer than 22 nucleotides in length and consists of 14, 15, 16, 17, 18, 19, 20, 21, or 22 nucleotides that are 100% complementary to a consecutive sequence within a 22 nucleotide target sequence that spans the most 3′ terminal 22 nucleotides of exon 50 and the most 5′ terminal 5 nucleotides of intron 50, together spanning a target domain of 27 nucleotides represented by SEQ ID NO:53 (underlined in FIG. 1). As outlined herein and in the accompanying examples, before the present invention was made, two AONs that yield skipping of exon 50 of human USH2A pre-mRNA were known from the prior art (WO 2016/005514). These two AONs are herein referred to as Radboud-1 and Radboud-2, or as AON20 and AON21 herein, respectively. As shown in the accompanying examples especially AON21 was able to quite sufficiently give exon 50 skipping from human USH2A pre-mRNA in transfection assays. Quite surprisingly, the inventors of the present invention identified a completely different region within the human USH2A sequence that could be targeted for exon 50 skipping wherein the AONs outperformed the oligonucleotides from the art. This region is located at the boundary of exon 50 and intron 50 and relates to a 27-nucleotide domain covering the 22 most 3′ located nucleotides in exon 50 and the 5 most 5′ located nucleotides in intron 50. Although AON21 outperformed the newly tested oligonucleotides in most instances, it turned out that such was only the case when transfection was used to deliver the oligonucleotides to the cells. The inventors surprisingly found that when no transfection was used, but rather left the oligonucleotides within the medium to enter the cells as is, without transfection (referred to as ‘gymnotic uptake’) the AONs targeting the newly identified domain were more efficient in exon 50 skipping than the best performing AON from the prior art. This shows that the inventors were able to identify new and unrelated AONs that were more efficient than the AONs of the prior art under more physiological conditions. AON21 is a 23-mer. The AONs of the present invention target a consecutive stretch of nucleotides within SEQ ID NO:53, and preferably are shorter than 23 nucleotides, more preferably 14, 15, 16, 17, 18, 19, 20, 21, or 22 nucleotides in length, more preferably 16, 17, 18, 19, 20, 21, or 22 nucleotides in length. Especially improved skipping efficiencies were found with AON15 (20-mer; SEQ ID NO:15), AON16 (20-mer), AON46 (21-mer; SEQ ID NO:46), AON47 (20-mer; SEQ ID NO:47), AON48 (19-mer; SEQ ID NO:48), and AON55 (SEQ ID NO:58). Even better skipping efficiency (using gymnotic uptake) was initially detected with AON48, but it is held here that all AONs that are shorter than 23 nucleotides and that target a consecutive sequence with SEQ ID NO:53 have such improved properties, supported by the results shown in FIGS. 5, 6, 7, and 8. In an experiment using gymnotic uptake it was found that AON55, AON56, AON103, AON48, AON54, AON53, AON15, AON70, AON50, AON78, AON100, AON101, AON69, AON65, AON68 and AON63 gave very high exon 50 skipping percentages upon gymnotic uptake (FIG. 6), which was strongly confirmed in an experiment using organoids, that is the best in vitro model and that is representative for the in vivo situation, and in which the AONs were also taken up in the cells without the aid of transfecting agents. These results clearly show that numerous AONs, especially those that target an area within SEQ ID NO:53 outperform the oligonucleotides from the prior art (exemplified by AON21).

The present invention therefore relates to an antisense oligonucleotide (AON) capable of skipping exon 50 from human USH2A pre-mRNA, wherein the AON comprises a 14 to 22 consecutive nucleotide sequence that is 100% complementary to a consecutive sequence within SEQ ID NO:53. Preferably, the AON according to the invention consists of 16, 17, 18, 19, 20, 21 or 22 nucleotides. In one particular aspect, the AON according to the invention consists of a sequence selected from the group consisting of SEQ ID NO:48, 15, 16, 45, 46, 47, 49, 50, 56 to 100, 54, 55, and 101 to 106 (herein referred to as AON48, AON15, AON16, AON45, AON46, AON47, AON49, AON50, and AON53 to AON105, respectively, see FIG. 1). Even more preferred are the compounds comprising, or consisting of the sequence of AON55, AON56, AON103, AON48, AON54, AON53, AON15, AON70, AON50, AON78, AON100, AON101, AON69, AON65, AON68 and AON63. These AONs are shorter than and outperform the AONs that were known from the art (such as AON21) and hybridize to a completely different region in the human USH2A pre-mRNA.

In a preferred aspect the AON is an oligoribonucleotide. In a further preferred aspect, the AON according to the invention comprises a 2′-0 alkyl modification, such as a 2′-O-methyl (2′-OMe) modified sugar. In a more preferred embodiment, all nucleotides in the AON are 2′-OMe modified. In another preferred aspect, the invention relates to an AON comprising a 2′-O-methoxyethyl (2′-methoxyethoxy, or 2′-MOE) modification. In a more preferred embodiment, all nucleotides of said AON carry a 2′-MOE modification. In yet another aspect the invention relates to an AON comprising at least one 2′-OMe and at least one 2′-MOE modification. In another preferred embodiment, the AON according to the present invention comprises at least one phosphorothioate (PS) modified linkage. In another preferred aspect, all sequential nucleotides are interconnected by PS linkages.

In yet another aspect, the invention relates to a viral vector expressing an AON according to the invention. The invention also relates to a pharmaceutical composition comprising an AON according to the invention or a viral vector according to the invention, and a pharmaceutically acceptable carrier.

In another aspect, the invention relates to an AON according to the invention, a viral vector according to the invention, or a pharmaceutical composition according to the invention for use in the treatment, prevention or delay of an USH2A-related disease or a condition requiring modulating splicing of USH2A pre-mRNA, such as Usher syndrome type II, preferably caused by a mutation selected from the group consisting of c.9799T>C, c.9958+69C>T, c.9815C>T, c.9811delA, and c.9958G>T. A preferred USH2A-related disease or condition is therefore one that is caused by a mutation in exon 50 of the human USH2A gene. In one aspect, the invention relates to an AON for use according to the invention, wherein the AON is for intravitreal administration and is dosed in an amount ranging from 5 μg to 500 μg of total AON per eye, preferably from 10 μg to 100 μg, more preferably from 25 μg to 100 μg. Preferably, the AON is administered in a naked form (as is, without being carried by a particle such as a nanoparticle or liposome), and preferably the administration to the vitreous is by injection. Preferably, the AON for use according to the invention is administered to the eye, wherein the AON is dosed in an amount ranging from 25 μg to 100 μg of total AON per eye, such as about 25, 30, 35, 40, 45, 50, 55, 60, 65, 70, 75, 80, 85, 90, 95, or 100 μg total AON per eye.

In another embodiment the invention relates to a use of an AON according to the invention, a viral vector according to the invention, or a pharmaceutical composition according to the invention for the preparation of a medicament for the treatment, prevention or delay of an USH2A-related disease or a condition requiring modulating splicing of USH2A pre-mRNA, such as Usher syndrome type II, preferably caused by a mutation selected from the group consisting of c.9799T>C, c.9958+69C>T, c.9815C>T, c.9811delA, and c.9958G>T.

In another embodiment, the invention relates to an in vitro, ex vivo or in vivo method for modulating splicing of USH2A pre-mRNA in a cell, comprising the steps of: administering to the cell an AON according to the invention, a viral vector according to the invention, or a pharmaceutical composition according to the invention; allowing the hybridization of the AON to its complementary sequence in USH2A target RNA molecule in the cell; and allowing the skip of exon 50 from the target RNA molecule. Optionally, the method further comprises the step of analyzing whether the skip of exon 50 from the USH2A target RNA molecule has occurred, which can be performed using methods as disclosed herein and/or by other methods generally known to the person skilled in the art. The invention also relates to a method for the treatment of a USH2A-related disease or condition requiring modulating splicing of USH2A pre-mRNA of an individual in need thereof, said method comprising contacting a cell of said individual with an AON according to the invention, a viral vector according to the invention, or a pharmaceutical composition according to the invention. Contacting the cell of the individual may be in vivo, by intravitreal injection of the AON to the patient in need thereof, or through ex vivo procedures, wherein treated cells, that have received the AON, viral vector or pharmaceutical composition, are transplanted back to the patient, thereby to treat the disease.

In all embodiments of the invention, the terms ‘modulating splicing’ and ‘exon skipping’ are synonymous. In respect of USH2A, ‘splice switching’, ‘modulating splicing’ or ‘exon skipping’ are to be construed as the exclusion of exon 50 from the resulting USH2A mRNA. In a preferred setting the exon 50 that needs to be skipped harbors unwanted mutations, leading to Usher syndrome, as outlined herein. For the purpose of the invention the terms ‘aberrant exon 50’ or ‘aberrant USH2A exon 50’ are synonymous and considered to mean the presence of a disease-causing mutation in exon 50 of the human USH2A gene. The term ‘exon skipping’ is herein defined as inducing, producing or increasing production within a cell of a mature mRNA that does not contain a particular exon (in the current case exon 50 of the human USH2A gene) that would be present in the mature mRNA without exon skipping. Exon skipping is achieved by providing a cell expressing the pre-mRNA of said mature mRNA with a molecule capable of interfering with sequences such as, for example, the (cryptic) splice donor or (cryptic) splice acceptor sequence required for allowing the enzymatic process of splicing, or with a molecule that is capable of interfering with an exon inclusion signal required for recognition of a stretch of nucleotides as an exon to be included in the mature mRNA; such molecules are herein referred to as ‘exon skipping molecules’, as ‘exon 50 skipping molecules’, as ‘AONs capable of skipping exon 50 from human USH2A pre-mRNA’, or as ‘exon skipping AONs’, and varieties thereof. The term ‘pre-mRNA’ refers to a non-processed or partly processed precursor mRNA that is synthesized from a DNA template of a cell by transcription, such as in the nucleus.

The terms ‘antisense oligonucleotide’, ‘oligonucleotide’, ‘single-stranded antisense oligonucleotide’, ‘AON’, ‘oligo’ and varieties thereof are used interchangeably herein and are understood to refer to a molecule with a nucleotide sequence that is substantially complementary to a target nucleotide sequence in a pre-mRNA molecule, hnRNA (heterogenous nuclear RNA) or mRNA molecule. The degree of complementarity (or substantial complementarity) of the antisense sequence is preferably such that a molecule comprising the antisense sequence can form a stable double stranded hybrid with the target nucleotide sequence in the RNA molecule under physiological conditions. Preferably, the AON has 100% complementarity with its target sequence. The skilled person understands that the AONs as exemplified herein have 100% complementarity with the wild type sequence of human USH2A, but in the event that the target sequence comprises a mutation which makes that the mutation exon 50 should be skipped, an AON of the present invention may be altered in sequence such that it has 100% complementarity to the mutant sequence. This is especially relevant for the present invention when the mutation in exon 50 is within its 3′ terminal end, and even more relevant when the mutation is within the terminal (3′) 22 nucleotides of exon 50. A non-limiting example of such a mutation is the c.9958G>T mutation affecting the last nucleotide (G) of exon 50. Hence, the present invention also relates to AONs that are 14-22 nt long and that have 100% complementarity to a mutant exon 50 sequence, when the mutation is in the last 14-22 nucleotides of the 3′ end of exon 50, respectively. Purely as a non-limiting example, AON48 (SEQ ID NO:48: 5′-ACC UGG AAG GCG AUU GUA C-3′) would have the sequence 5′-ACA UGG AAG GCG AUU GUA C-3′ (SEQ ID NO:108; AON48*) when targeting the USH2A pre-mRNA comprising the c.9958C>T mutation. AONs that are 100% complementary to a mutant USH2A pre-mRNA, but that differ at the mutation position from any AONs as disclosed herein (and that were disclosed and/or used and/or tested on wild type USH2A pre-mRNA for proper exon 50 skip testing) are considered equivalent to the AONs that are 100% complementary to the wild type sequence, and are within the scope of the present invention.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the elements is present, unless the context clearly requires that there be one and only one of the elements. The indefinite article “a” or “an” thus usually means “at least one”. The word “about” or “approximately” when used in association with a numerical value (e.g. about 10) preferably means that the value may be the given value (of 10) plus or minus 0.1% of the value.

In one embodiment, an exon 50 skipping molecule as defined herein is an AON that binds and/or is complementary to a specified target RNA sequence within a target RNA molecule, preferably a target pre-mRNA molecule. Binding to one of the specified target sequences, preferably in the context of a mutated USH2A exon 50 may be assessed via techniques known to the skilled person. A preferred technique is gel mobility shift assay as described in EP1619249. In a preferred embodiment, an exon 50 skipping AON is said to bind to one of the specified sequences as soon as a binding of said molecule to a labeled target sequence is detectable in a gel mobility shift assay.

In all embodiments of the invention, an exon 50 skipping molecule is preferably an AON. Preferably, an exon 50 skipping AON according to the invention is an AON, which is at least 90%, and more preferably 100% complementary to a 14-22 nucleotide consecutive sequence of SEQ ID NO:53.

The term ‘substantially complementary’ used in the context of the invention indicates that some mismatches in the antisense sequence are allowed if the functionality, i.e. inducing skipping of the mutated USH2A exon 50 is still acceptable. Preferably, the complementarity is from 90% to 100%. In general, this allows for 1 or 2 mismatches in an AON of 20 nucleotides. As outlined above already, the skilled person understands that an AON may be 100% complementary to a sequence harboring a mutation, which means that it is not 100% complementary to the corresponding wild type sequence, although it may be still active in causing exon 50 skipping in both wild type and mutant settings.

The invention provides a method for designing an exon 50 skipping AON able to induce skipping of the mutated USH2A exon 50. First, the AON is selected to bind to and/or to be complementary to exon 50, possibly with stretches of the flanking intron sequences (see FIG. 1). Preferably, the exon skipping AON has acceptable RNA binding kinetics and/or thermodynamic properties. The RNA binding kinetics and/or thermodynamic properties are at least in part determined by the melting temperature of an AON (Tm), and/or the free energy of the AON-target exon complex, applying methods known to the person skilled in the art. If a Tm is too high, the AON is expected to be less specific. An acceptable Tm and free energy depend on the sequence of the AON. Therefore, it is difficult to give preferred ranges for each of these parameters. An acceptable Tm may be ranged between 35 and 70° C. and an acceptable free energy may be ranged between 15 and 45 kcal/mol.

An AON of the invention is preferably one that can exhibit an acceptable level of functional activity. A functional activity of said AON is preferably to induce the skipping of the mutant USH2A exon 50 to a certain acceptable level, to provide an individual with a functional usherin protein and/or USH2A mRNA and/or at least in part decreasing the production of an aberrant usherin protein and/or mRNA. In a preferred embodiment, an AON is said to induce skipping of the mutated USH2A exon 50, when the mutated USH2A exon 50 skipping percentage as measured by digital-droplet PCR (ddPCR) is at least 2%, or at least 5%, or at least 10%, or at least 15%, or at least 20%, or at least 50%, or at least 55%, or at least 60%, or at least 65%, or at least 70%, or at least 75%, or at least 80%, or at least 85%, or at least 90%, or at least 95%, or 100% as compared to a control RNA product not treated with an AON or a negative control AON. These percentages may differ when cells are transfected with the AONs of interest or when gymnotic uptake is being applied. Assays to determine exon skipping and/or exon retention are described in the examples herein and may be supplemented with techniques known to the person skilled in the art.

As stated above, it is not absolutely required that all the bases in the region of complementarity are capable of pairing with bases in the opposing strand. For instance, when designing the AON, one may want to incorporate for instance a residue that does not base pair with the base on the complementary strand. Mismatches may, to some extent, be allowed, if under the circumstances in the cell, the stretch of nucleotides is sufficiently capable of hybridizing to the complementary part. In this context, ‘sufficiently’ preferably means that using a gel mobility shift assay as described in example 1 of EP1619249, binding of an AON is detectable.

Optionally, said AON may further be tested by delivery to retina cells of patients, by incubating the AONs directly with so-called eye-cups, which are ex vivo generated eye models (‘organoids’ generally generated from patient's cells), or by intravitreal injection in an animal model, or by intravitreal administration in human patients in the course of performing clinical trials. Skipping of targeted exon 50 may be assessed by RT-PCR or by ddPCR. The complementary regions are preferably designed such that, when combined, they are specific for the exon (yes/no including intron) sequences in the pre-mRNA. Such specificity may be created with various lengths of complementary regions as this depends on the actual sequences in other (pre-) mRNA molecules in the system. The risk that the AON also will be able to hybridize to one or more other pre-mRNA molecules decreases with increasing size of the AON. It is clear that AONs comprising mismatches in the region of complementarity but that retain the capacity to hybridize and/or bind to the targeted region(s) in the pre-mRNA, can be used according to the invention. However, preferably at least the complementary parts do not comprise such mismatches as AONs lacking mismatches in the complementary part typically generally have a higher efficiency and a higher specificity than AONs having such mismatches in one or more complementary regions. Without wishing to be bound by theory, it is thought that higher hybridization strengths (i.e. increasing number of interactions with the opposing strand) are favorable in increasing the efficiency of the process of interfering with the splicing machinery of the system. Preferably, the complementarity is from 90% to 100%.

An exon skipping AON of the invention is preferably an isolated single stranded molecule in the absence of its (target) counterpart sequence. An exon skipping AON of the invention is at least complementary to, and under physiological conditions binds to a consecutive stretch of nucleotides within sequence SEQ ID NO:53. It will be understood that an exon 50 skipping AON does not have to be complementary to the exact position in exon 50 that is mutated. It may be that the AON is complementary to the wild type exon 50 sequence (possibly with its surrounding intron sequences), while still being able to give exon 50 skipping. The aim is to skip a mutated exon 50 from USH2A pre-mRNA, not to have an AON that specifically targets a region containing the mutation, although such is not explicitly excluded. Any mutation in USH2A exon 50 that causes disease (such as Usher syndrome) is preferably removed from the final mRNA (and the resulting protein) by using an AON of the present invention, wherein the sequence of the AON may be complementary to a non-mutated region. The invention relates to AONs that may be fully complementary to the wild type target sequence but may also be adjusted in sequence to become 100% complementary to a mutant sequence, if the mutation is in the region of AON complementarity. In that particular case the AON is substantially complementary to the mutant sequence and may then differ from the wild type sequences of the AONs that are generally referred to herein. The invention is generally explained for any mutation that may be present in exon 50 of human USH2A, but specific mutations may be targeted by AONs that are (preferably 100%) complementary to that specific mutation and its surrounding sequences, 5′ and/or 3′ from the mutation.

An exon 50 skipping AON according to the invention may contain one of more RNA residues, or one or more DNA residues, and/or one or more nucleotide analogues or equivalents, as will be further detailed herein below. It is preferred that an exon 50 skipping AON of the invention comprises one or more residues that are modified by non-naturally occurring modifications to increase nuclease resistance, and/or to increase the affinity of the AON for the target sequence, especially when the AON is administered in a naked from, i.e. not in association with a delivery vehicle such as a (viral) vector. Therefore, in a preferred embodiment, the AON sequence comprises at least one nucleotide analogue or equivalent, wherein a nucleotide analogue or equivalent is defined as a residue having a modified base, and/or a modified backbone, and/or a non-natural internucleoside linkage, or a combination of these modifications.

The skilled person knows that an oligonucleotide, such as an RNA oligonucleotide, generally consists of repeating monomers. Such a monomer is most often a nucleotide or a nucleotide analogue. The most common naturally occurring nucleotides in RNA are adenosine monophosphate (A), cytidine monophosphate (C), guanosine monophosphate (G), and uridine monophosphate (U). These consist of a pentose sugar, a ribose, a 5′-linked phosphate group which is linked via a phosphate ester, and a 1′-linked base. The sugar connects the base and the phosphate and is therefore often referred to as the “scaffold” of the nucleotide. A modification in the pentose sugar is therefore often referred to as a “scaffold modification”. For severe modifications, the original pentose sugar might be replaced in its entirety by another moiety that similarly connects the base and the phosphate. It is therefore understood that while a pentose sugar is often a scaffold, a scaffold is not necessarily a pentose sugar.

A base, sometimes called a nucleobase, is generally adenine, cytosine, guanine, thymine or uracil, or a derivative thereof. Cytosine, thymine and uracil are pyrimidine bases, and are generally linked to the scaffold through their 1-nitrogen. Adenine and guanine are purine bases and are generally linked to the scaffold through their 9-nitrogen.

A nucleotide is generally connected to neighboring nucleotides through condensation of its 5′-phosphate moiety to the 3′-hydroxyl moiety of the neighboring nucleotide monomer. Similarly, its 3′-hydroxyl moiety is generally connected to the 5′-phosphate of a neighboring nucleotide monomer. This forms phosphodiester bonds. The phosphodiesters and the scaffold form an alternating copolymer. The bases are grafted on this copolymer, namely to the scaffold moieties. Because of this characteristic, the alternating copolymer formed by linked monomers of an oligonucleotide is often called the “backbone” of the oligonucleotide. Because phosphodiester bonds connect neighboring monomers together, they are often referred to as “backbone linkages”. It is understood that when a phosphate group is modified so that it is instead an analogous moiety such as a phosphorothioate, such a moiety is still referred to as the backbone linkage of the monomer. This is referred to as a “backbone linkage modification”. In general terms, the backbone of an oligonucleotide comprises alternating scaffolds and backbone linkages.

In one aspect, the nucleobase in an AON of the present invention is adenine, cytosine, guanine, thymine, or uracil. In another aspect, the nucleobase is a modified form of adenine, cytosine, guanine, or uracil. In another aspect, the modified nucleobase is hypoxanthine (the nucleobase in inosine), pseudouracil, pseudocytosine, 1-methylpseudouracil, orotic acid, agmatidine, lysidine, 2-thiouracil, 2-thiothymine, 5-halouracil, 5-halomethyluracil, 5-trifluoromethyluracil, 5-propynyluracil, 5-propynylcytosine, 5-aminomethyluracil, 5-hydroxymethyluracil, 5-formyluracil, 5-am inomethylcytosine, 5-formylcytosine, 5-hydroxymethylcytosine, 7-deazaguanine, 7-deazaadenine, 7-deaza-2,6-diaminopurine, 8-aza-7-deazaguanine, 8-aza deazaadenine, 8-aza-7-deaza-2,6-diaminopurine, pseudoisocytosine, N4-ethylcytosine, N2-cyclopentylguanine, N2-cyclopentyl-2-aminopurine, N2-propyl-2-aminopurine, 2,6-diaminopurine, 2-aminopurine, G-clamp, Super A, Super T, Super G, amino-modified nucleobases or derivatives thereof; and degenerate or universal bases, like 2,6-difluorotoluene, or absent like abasic sites (e.g. 1-deoxyribose, 1,2-dideoxyribose, 1-deoxy-2-O-methylribose, azaribose). The terms ‘adenine’, ‘guanine’, ‘cytosine’, ‘thymine’, ‘uracil’ and ‘hypoxanthine’ as used herein refer to the nucleobases as such. The terms ‘adenosine’, ‘guanosine’, ‘cytidine’, ‘thymidine’, ‘uridine’ and ‘inosine’ refer to the nucleobases linked to the (deoxy)ribosyl sugar. The term ‘nucleoside’ refers to the nucleobase linked to the (deoxy)ribosyl sugar. The term ‘nucleotide’ refers to the respective nucleobase-(deoxy)ribosyl-phospholinker, as well as any chemical modifications of the ribose moiety or the phospho group. Thus the term would include a nucleotide including a locked ribosyl moiety (comprising a 2′-4′ bridge, comprising a methylene group or any other group, well known in the art), a nucleotide including a linker comprising a phosphodiester, phosphotriester, phosphoro(di)thioate, methylphosphonates, phosphoramidate linkers, and the like. The sugar moiety can be a pyranose or derivative thereof, or a deoxypyranose or derivative thereof, preferably ribose or derivative thereof, or deoxyribose or derivative thereof. A preferred derivatized sugar moiety comprises a Locked Nucleic Acid (LNA), in which the 2′-carbon atom is linked to the 3′ or 4′ carbon atom of the sugar ring thereby forming a bicyclic sugar moiety. A preferred LNA comprises 2′-0, 4′-C-ethylene-bridged nucleic acid (Morita et al. 2001. Nucleic Acid Res Supplement No. 1:241-242).

Sometimes the terms adenosine and adenine, guanosine and guanine, cytosine and cytidine, uracil and uridine, thymine and thymidine, inosine and hypoxanthine, are used interchangeably to refer to the corresponding nucleobase, nucleoside or nucleotide. Sometimes the terms nucleobase, nucleoside and nucleotide are used interchangeably, unless the context clearly requires differently. Modified bases comprise synthetic and natural bases such as inosine, xanthine, hypoxanthine and other -aza, deaza, -hydroxy, -halo, -thio, thiol, -alkyl, -alkenyl, -alkynyl, thioalkyl derivatives of pyrimidine and purine bases that are or will be known in the art.

In one aspect, an AON of the present invention comprises a 2′-substituted phosphorothioate monomer, preferably a 2′-substituted phosphorothioate RNA monomer, a 2′-substituted phosphate RNA monomer, or comprises 2′-substituted mixed phosphate/phosphorothioate monomers. It is noted that DNA is considered as an RNA derivative in respect of 2′ substitution. An AON of the present invention comprises at least one 2′-substituted RNA monomer connected through or linked by a phosphorothioate or phosphate backbone linkage, or a mixture thereof. The 2′-substituted RNA preferably is 2′-F, 2′-H (DNA), 2′-O-Methyl or 2′-O-(2-methoxyethyl). The 2′-O-Methyl is often abbreviated to “2′-OMe” and the 2′-O-(2-methoxyethyl) moiety is often abbreviated to “2′-MOE”. In a preferred embodiment of this aspect is provided an AON according to the invention, wherein the 2′-substituted monomer can be a 2′-substituted RNA monomer, such as a 2′-F monomer, a 2′—NH₂ monomer, a 2′-H monomer (DNA), a 2′-O-substituted monomer, a 2′-OMe monomer or a 2′-MOE monomer or mixtures thereof. Preferably, any other 2′-substituted monomer within the AON is a 2′-substituted RNA monomer, such as a 2′-OMe RNA monomer or a 2′-MOE RNA monomer, which may also appear within the AON in combination.

Throughout the application, a 2′-OMe monomer within an AON of the present invention may be replaced by a 2′-OMe phosphorothioate RNA, a 2′-OMe phosphate RNA or a 2′-OMe phosphate/phosphorothioate RNA. Throughout the application, a 2′-MOE monomer may be replaced by a 2′-MOE phosphorothioate RNA, a 2′-MOE phosphate RNA or a 2′-MOE phosphate/phosphorothioate RNA. Throughout the application, an oligonucleotide consisting of 2′-OMe RNA monomers linked by or connected through phosphorothioate, phosphate or mixed phosphate/phosphorothioate backbone linkages may be replaced by an oligonucleotide consisting of 2′-OMe phosphorothioate RNA, 2′-OMe phosphate RNA or 2′-OMe phosphate/phosphorothioate RNA. Throughout the application, an oligonucleotide consisting of 2′-MOE RNA monomers linked by or connected through phosphorothioate, phosphate or mixed phosphate/phosphorothioate backbone linkages may be replaced by an oligonucleotide consisting of 2′-MOE phosphorothioate RNA, 2′-MOE phosphate RNA or 2′-MOE phosphate/phosphorothioate RNA.

In addition to the specific preferred chemical modifications at certain positions in compounds of the invention, compounds of the invention may comprise or consist of one or more (additional) modifications to the nucleobase, scaffold and/or backbone linkage, which may or may not be present in the same monomer, for instance at the 3′ and/or 5′ position. A scaffold modification indicates the presence of a modified version of the ribosyl moiety as naturally occurring in RNA (i.e. the pentose moiety), such as bicyclic sugars, tetrahydropyrans, hexoses, morpholinos, 2′-modified sugars, 4′-modified sugar, 5′-modified sugars and 4′-substituted sugars. Examples of suitable modifications include, but are not limited to 2′-O-modified RNA monomers, such as 2′-O-alkyl or 2′-O-(substituted)alkyl such as 2′-O-methyl, 2′-O-(2-cyanoethyl), 2′-MOE, 2′-O-(2-thiomethyl)ethyl, 2′-O-butyryl, 2′-O-propargyl, 2′-O-allyl, 2′-O-(2-aminopropyl), 2′-O-(2-(dimethylamino)propyl), 2′-O-(2-amino)ethyl, 2′-O-(2-(dimethylamino)ethyl); 2′-deoxy (DNA); 2′-O-(haloalkyl)methyl such as 2′-O-(2-chloroethoxy)methyl (MCEM), 2′-O-(2,2-dichloroethoxy)methyl (DCEM); 2′-O-alkoxycarbonyl such as 2′-O-[2-(methoxycarbonyl)ethyl] (MOCE), 2′-O-[2-N-methylcarbamoyl)ethyl] (MCE), 2′-O-[2-(N,N-dimethylcarbamoyl)ethyl] (DCME); 2′-halo e.g. 2′-F, FANA; 2′-O-[2-(methylamino)-2-oxoethyl] (NMA); a bicyclic or bridged nucleic acid (BNA) scaffold modification such as a conformationally restricted nucleotide (CRN) monomer, a locked nucleic acid (LNA) monomer, a xylo-LNA monomer, an α-LNA monomer, an α-L-LNA monomer, a β-D-LNA monomer, a 2′-amino-LNA monomer, a 2′-(alkylamino)-LNA monomer, a 2′-(acylamino)-LNA monomer, a 2′-N-substituted 2′-amino-LNA monomer, a 2′-thio-LNA monomer, a (2′-0,4′-C) constrained ethyl (cEt) BNA monomer, a (2′-O,4′-C) constrained methoxyethyl (cMOE) BNA monomer, a 2′,4′-BNA^{NC} (NH) monomer, a 2′,4′-BNA^{NC} (NMe) monomer, a 2′,4′-BNA^{NC} (NBn) monomer, an ethylene-bridged nucleic acid (ENA) monomer, a carba-LNA (cLNA) monomer, a 3,4-dihydro-2H-pyran nucleic acid (DpNA) monomer, a 2′-C-bridged bicyclic nucleotide (CBBN) monomer, an oxo-CBBN monomer, a heterocyclic-bridged BNA monomer (such as triazolyl or tetrazolyl-linked), an amido-bridged BNA monomer (such as AmNA), an urea-bridged BNA monomer, a sulfonamide-bridged BNA monomer, a bicyclic carbocyclic nucleotide monomer, a TriNA monomer, an α-L-TriNA monomer, a bicyclo DNA (bcDNA) monomer, an F-bcDNA monomer, a tricyclo DNA (tcDNA) monomer, an F-tcDNA monomer, an alpha anomeric bicyclo DNA (abcDNA) monomer, an oxetane nucleotide monomer, a locked PMO monomer derived from 2′-amino LNA, a guanidine-bridged nucleic acid (GuNA) monomer, a spirocyclopropylene-bridged nucleic acid (scpBNA) monomer, and derivatives thereof; cyclohexenyl nucleic acid (CeNA) monomer, altriol nucleic acid (ANA) monomer, hexitol nucleic acid (HNA) monomer, fluorinated HNA (F-HNA) monomer, pyranosyl-RNA (p-RNA) monomer, 3′-deoxypyranosyl DNA (p-DNA), unlocked nucleic acid UNA); an inverted version of any of the monomers above.

A “backbone modification” indicates the presence of a modified version of the ribosyl moiety (“scaffold modification”), as indicated above, and/or the presence of a modified version of the phosphodiester as naturally occurring in RNA (“backbone linkage modification”). Examples of internucleoside linkage modifications are phosphorothioate (PS), chirally pure phosphorothioate, Rp phosphorothioate, Sp phosphorothioate, phosphorodithioate (PS2), phosphonoacetate (PACE), thophosphonoacetate, phosphonacetamide (PACA), thiophosphonacetamide, phosphorothioate prodrug, S-alkylated phosphorothioate, H-phosphonate, methyl phosphonate, methyl phosphonothioate, methyl phosphate, methyl phosphorothioate, ethyl phosphate, ethyl phosphorothioate, boranophosphate, boranophosphorothioate, methyl boranophosphate, methyl boranophosphorothioate, methyl boranophosphonate, methyl boranophosphonothioate, phosphoryl guanidine (PGO), methylsulfonyl phosphoroamidate, phosphoramidite, phosphonamidite, N3′→P5′ phosphoramidate, N3′→P5′ thiophosphoramidate, phosphorodiamidate, phosphorothiodiamidate, sulfamate, dimethylenesulfoxide, sulfonate, triazole, oxalyl, carbamate, methyleneimino (MMI), and thioacetamido (TANA); and their derivatives.

The present invention also relates to a chirally enriched population of modified AONs according to the invention, wherein the population is enriched for modified AONs comprising at least one particular phosphorothioate internucleoside linkage having a particular stereochemical configuration, preferably wherein the population is enriched for modified AONs comprising at least one particular phosphorothioate internucleoside linkage having the Sp configuration, or wherein the population is enriched for modified AONs comprising at least one particular phosphorothioate internucleoside linkage having the Rp configuration.

In a preferred embodiment, the nucleotide analogue or equivalent comprises a modified backbone, exemplified by morpholino backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones. Phosphorodiamidate morpholino oligomers are modified backbone oligonucleotides that have previously been investigated as antisense agents. Morpholino oligonucleotides have an uncharged backbone in which the deoxyribose sugar of DNA is replaced by a six membered ring and the phosphodiester linkage is replaced by a phosphorodiamidate linkage. Morpholino oligonucleotides are resistant to enzymatic degradation and appear to function as antisense agents by arresting translation or interfering with pre-mRNA splicing rather than by activating RNase H. Morpholino oligonucleotides have been successfully delivered to tissue culture cells by methods that physically disrupt the cell membrane, and one study comparing several of these methods found that scrape loading was the most efficient method of delivery; however, because the morpholino backbone is uncharged, cationic lipids are not effective mediators of morpholino oligonucleotide uptake in cells.

It is further preferred that the linkage between the residues in a backbone do not include a phosphorus atom, such as a linkage that is formed by short chain alkyl or cycloalkyl internucleoside linkages, mixed heteroatom and alkyl or cycloalkyl internucleoside linkages, or one or more short chain heteroatomic or heterocyclic internucleoside linkages.

A preferred nucleotide analogue or equivalent comprises a Peptide Nucleic Acid (PNA), having a modified polyamide backbone (Nielsen, et al. (1991) Science 254, 1497-1500). PNA-based molecules are true mimics of DNA molecules in terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer. Since the backbone of a PNA molecule contains no charged phosphate groups, PNA-RNA hybrids are usually more stable than RNA-RNA or RNA-DNA hybrids, respectively (Egholm et al. (1993) Nature 365:566-568).

It is understood by a skilled person that it is not necessary for all positions in an AON to be modified uniformly. In addition, more than one of the analogues or equivalents may be incorporated in a single AON or even at a single position within an AON. In certain embodiments, an AON of the invention has at least two different types of analogues or equivalents. A preferred exon skipping AON according to the invention comprises a 2′-0 alkyl phosphorothioated antisense oligonucleotide, such as 2′-OMe modified ribose (RNA), 2′-O-ethyl modified ribose, 2′-O-propyl modified ribose, and/or substituted derivatives of these modifications such as halogenated derivatives. An effective AON according to the invention comprises a 2′-OMe ribose and/or a 2′-MOE ribose with a (preferably full) phosphorothioated backbone.

It will also be understood by a skilled person that different AONs can be combined for efficiently skipping of the aberrant USH2A exon 50. In a preferred embodiment, a combination of at least two AONs are used in a method of the invention, such as 2, 3, 4, or 5 different AONs. Hence, the invention also relates to a composition comprising a set of AONs comprising at least one AON according to the present invention, optionally further comprising AONs as disclosed herein.

An AON of the present invention can be linked to a moiety that enhances uptake of the AON in cells, preferably retina cells. Examples of such moieties are cholesterols, carbohydrates, vitamins, biotin, lipids, phospholipids, cell-penetrating peptides including but not limited to antennapedia, TAT, transportan and positively charged amino acids such as oligoarginine, poly-arginine, oligolysine or polylysine, antigen-binding domains such as provided by an antibody, a Fab fragment of an antibody, or a single chain antigen binding domain such as a cameloid single domain antigen-binding domain.

An exon 50 skipping AON according to the invention may be indirectly administrated using suitable means known in the art. It may for example be provided to an individual or a cell, tissue or organ of said individual in the form of an expression vector wherein the expression vector encodes a transcript comprising said oligonucleotide. The expression vector may be introduced into a cell, tissue, organ or individual via a gene delivery vehicle. In a preferred embodiment, there is provided a viral-based expression vector comprising an expression cassette or a transcription cassette that drives expression or transcription of an AON as identified herein. Accordingly, the invention provides a viral vector expressing an exon 50 skipping AON according to the invention when placed under conditions conducive to expression of the exon 50 skipping AON. A cell can be provided with an exon skipping molecule capable of interfering with essential sequences that result in highly efficient skipping of the aberrant USH2A exon 50 by plasmid-derived AON expression or viral expression provided by adenovirus- or adeno-associated virus-based vectors. Expression may be driven by a polymerase II-promoter (Pol II) such as a U7 promoter or a polymerase III (Pol III) promoter, such as a U6 RNA promoter. A preferred delivery vehicle is a viral vector such as an adeno associated virus vector (AAV), or a retroviral vector such as a lentivirus vector and the like. Also, plasmids, artificial chromosomes, plasmids usable for targeted homologous recombination and integration in the human genome of cells may be suitably applied for delivery of an oligonucleotide as defined herein. Preferred for the current invention are those vectors wherein transcription is driven from Pol III promoters, and/or wherein transcripts are in the form fusions with U1 or U7 transcripts, which yield good results for delivering small transcripts. It is within the skill of the artisan to design suitable transcripts. Preferred are Pol III driven transcripts, preferably, in the form of a fusion transcript with an U1 or U7 transcript. Such fusions may be generated as described (Gorman et al. 1998. Proc Natl Acad Sci USA 95(9):4929-34; Suter et al. 1999. Hum Mol Genet 8(13):2415-23).

The exon 50 skipping AON may be delivered in association with a viral vector. Typically, this is in the form of an RNA transcript that comprises the sequence of an oligonucleotide according to the invention in a part of the transcript. An AAV vector according to the invention is a recombinant AAV vector and refers to an AAV vector comprising part of an AAV genome comprising an encoded exon 50 skipping AON according to the invention encapsidated in a protein shell of capsid protein derived from an AAV serotype as depicted elsewhere herein. Part of an AAV genome may contain the inverted terminal repeats (ITR) derived from an adeno-associated virus serotype, such as AAV1, AAV2, AAV3, AAV4, AAV5, AAV6, AAV7, AAV8, AAV9 and others. Protein shell comprised of capsid protein may be derived from an AAV serotype such as AAV1, 2, 3, 4, 5, 6, 7, 8, 9 and others. A protein shell may also be named a capsid protein shell. AAV vector may have one or preferably all wild type AAV genes deleted but may still comprise functional ITR nucleic acid sequences. Functional ITR sequences are necessary for the replication, rescue and packaging of AAV virions. The ITR sequences may be wild type sequences or may have at least 80%, 85%, 90%, 95, or 100% sequence identity with wild type sequences or may be altered by for example in insertion, mutation, deletion or substitution of nucleotides, as long as they remain functional. In this context, functionality refers to the ability to direct packaging of the genome into the capsid shell and then allow for expression in the host cell to be infected or target cell. In the context of the invention a capsid protein shell may be of a different serotype than the AAV vector genome ITR. An AAV vector according to present the invention may thus be composed of a capsid protein shell, i.e. the icosahedral capsid, which comprises capsid proteins (VP1, VP2, and/or VP3) of one AAV serotype, e.g. AAV serotype 2, whereas the ITRs sequences contained in that AAV5 vector may be any of the AAV serotypes described above, including an AAV2 vector. An “AAV2 vector” thus comprises a capsid protein shell of AAV serotype 2, while e.g. an “AAV5 vector” comprises a capsid protein shell of AAV serotype 5, whereby either may encapsidate any AAV vector genome ITR according to the invention. Preferably, a recombinant AAV vector according to the invention comprises a capsid protein shell of AAV serotype 2, 5, 8 or AAV serotype 9 wherein the AAV genome or ITRs present in said AAV vector are derived from AAV serotype 2, 5, 8 or AAV serotype 9; such AAV vector is referred to as an AAV2/2, AAV 2/5, AAV2/8, AAV2/9, AAV5/2, AAV5/5, AAV5/8, AAV 5/9, AAV8/2, AAV 8/5, AAV8/8, AAV8/9, AAV9/2, AAV9/5, AAV9/8, or an AAV9/9 vector.

More preferably, a recombinant AAV vector according to the invention comprises a capsid protein shell of AAV serotype 2 and the AAV genome or ITRs present in said vector are derived from AAV serotype 5; such vector is referred to as an AAV 2/5 vector. More preferably, a recombinant AAV vector according to the invention comprises a capsid protein shell of AAV serotype 2 and the AAV genome or ITRs present in said vector are derived from AAV serotype 8; such vector is referred to as an AAV 2/8 vector. More preferably, a recombinant AAV vector according to the invention comprises a capsid protein shell of AAV serotype 2 and the AAV genome or ITRs present in said vector are derived from AAV serotype 9; such vector is referred to as an AAV 2/9 vector. More preferably, a recombinant AAV vector according to the invention comprises a capsid protein shell of AAV serotype 2 and the AAV genome or ITRs present in said vector are derived from AAV serotype 2; such vector is referred to as an AAV 2/2 vector. A nucleic acid molecule encoding an exon 50 skipping AON according to the invention represented by a nucleic acid sequence of choice is preferably inserted between the AAV genome or ITR sequences as identified above, for example an expression construct comprising an expression regulatory element operably linked to a coding sequence and a 3′ termination sequence. “AAV helper functions” generally refers to the corresponding AAV functions required for AAV replication and packaging supplied to the AAV vector in trans. AAV helper functions complement the AAV functions which are missing in the AAV vector, but they lack AAV ITRs (which are provided by the AAV vector genome). AAV helper functions include the two major ORFs of AAV, namely the rep coding region and the cap coding region or functional substantially identical sequences thereof. Rep and Cap regions are well known in the art. The AAV helper functions can be supplied on an AAV helper construct, which may be a plasmid.

Introduction of the helper construct into the host cell can occur e.g. by transformation, transfection, or transduction prior to or concurrently with the introduction of the AAV genome present in the AAV vector as identified herein. The AAV helper constructs of the invention may thus be chosen such that they produce the desired combination of serotypes for the AAV vector's capsid protein shell on the one hand and for the AAV genome present in said AAV vector replication and packaging on the other hand. “AAV helper virus” provides additional functions required for AAV replication and packaging. Suitable AAV helper viruses include adenoviruses, herpes simplex viruses (such as HSV types 1 and 2) and vaccinia viruses. The additional functions provided by the helper virus can also be introduced into the host cell via vectors, as described in U.S. Pat. No. 6,531,456 incorporated herein by reference. Preferably, an AAV genome as present in a recombinant AAV vector according to the invention does not comprise any nucleotide sequences encoding viral proteins, such as the rep (replication) or cap (capsid) genes of AAV. An AAV genome may further comprise a marker or reporter gene, such as a gene for example encoding an antibiotic resistance gene, a fluorescent protein (e.g. gfp) or a gene encoding a chemically, enzymatically or otherwise detectable and/or selectable product (e.g. lacZ, aph, etc.) known in the art. Preferably, an AAV vector according to the invention is constructed and produced according to the methods in the examples herein. A preferred AAV vector according to the invention is an AAV vector, preferably an AAV2/5, AAV2/8, AAV2/9 or AAV2/2 vector, expressing an USH2A exon 50 skipping AON according to the invention. A further preferred AAV vector according to the invention is an AAV vector, preferably an AAV2/5, AAV2/8, AAV2/9 or AAV2/2 vector, expressing an exon 50 skipping AON according to the invention that is complementary to a consecutive sequence within SEQ ID NO:53.

Improvements in means for providing an individual or a cell, tissue, organ of said individual with an exon 50 skipping AON according to the invention, are anticipated considering the progress that has already thus far been achieved. Such future improvements may of course be incorporated to achieve the mentioned effect on restructuring of mRNA using a method of the invention. An exon 50 skipping AON according to the invention can be delivered as is to an individual, a cell, tissue or organ of said individual. When administering an exon 50 skipping AON according to the invention, it is preferred that the AON is dissolved in a solution that is compatible with the delivery method. Retina or inner ear cells can be provided with a plasmid for AON expression by providing the plasmid in an aqueous solution. Alternatively, a preferred delivery method for an AON or a plasmid for AON expression is a viral vector or nanoparticles. Preferably viral vectors or nanoparticles are delivered to retina or inner ear cells. Such delivery to retina or inner ear cells or other relevant cells may be in vivo, in vitro or ex vivo. Nanoparticles and micro particles that may be used for in vivo AON delivery are well known in the art. Alternatively, a plasmid can be provided by transfection using known transfection reagents. For intraventricular administration it is preferred that the solution is a physiological salt solution. Particularly preferred in the invention is the use of an excipient or transfection reagents that will aid in delivery of each of the constituents as defined herein to a cell and/or into a cell (preferably a retina cell). Preferred are excipients or transfection reagents capable of forming complexes, nanoparticles, micelles, vesicles and/or liposomes that deliver each constituent as defined herein, complexed or trapped in a vesicle or liposome through a cell membrane. Many of these excipients are known in the art. Suitable excipients or transfection reagents comprise polyethylenimine (PEI; ExGen500 (MBI Fermentas)), LipofectAMINE™ 2000 (Invitrogen) or derivatives thereof, or similar cationic polymers, including polypropyleneimine or polyethylenimine copolymers (PECs) and derivatives, synthetic amphiphils (SAINT-18), lipofectin™, DOTAP and/or viral capsid proteins that are capable of self-assembly into particles that can deliver each constituent as defined herein to a cell, preferably a retina cell. Such excipients have been shown to efficiently deliver an AON to a wide variety of cultured cells, including retina cells. Their high transfection potential is combined with an excepted low to moderate toxicity in terms of overall cell survival. The ease of structural modification can be used to allow further modifications and the analysis of their further (in vivo) nucleic acid transfer characteristics and toxicity. Lipofectin represents an example of a liposomal transfection agent. It consists of two lipid components, a cationic lipid N-[1-(2,3 dioleoyloxy)propyl]-N, N, N-trimethylammonium chloride (DOTMA) (cp. DOTAP which is the methylsulfate salt) and a neutral lipid dioleoylphosphatidyl ethanolamine (DOPE). The neutral component mediates the intracellular release. Another group of delivery system are polymeric nanoparticles. Polycations such as diethylamino ethylaminoethyl (DEAE)-dextran, which are well known as DNA transfection reagent can be combined with butylcyanoacrylate (PBCA) and hexylcyanoacrylate (PHCA) to formulate cationic nanoparticles that can deliver AONs across cell membranes into cells. In addition to these common nanoparticle materials, the cationic peptide protamine offers an alternative approach to formulate an oligonucleotide with colloids. This colloidal nanoparticle system can form so called proticles, which can be prepared by a simple self-assembly process to package and mediate intracellular release of an AON. The skilled person may select and adapt any of the above or other commercially available alternative excipients and delivery systems to package and deliver an exon skipping molecule for use in the current invention to deliver it for the prevention, treatment or delay of a USH2A related disease or condition.

“Prevention, treatment or delay of a USH2A related disease or condition” is herein preferably defined as preventing, halting, ceasing the progression of, or reversing partial or complete visual impairment or blindness, as well as preventing, halting, ceasing the progression of or reversing partial or complete auditory impairment or deafness that is caused by a genetic defect in the USH2A gene.

In addition, an exon 50 skipping AON according to the invention could be covalently or non-covalently linked to a targeting ligand specifically designed to facilitate the uptake into the cell, cytoplasm and/or its nucleus. Such ligand could comprise (i) a compound (including but not limited to peptide(-like) structures) recognizing cell, tissue or organ specific elements facilitating cellular uptake and/or (ii) a chemical compound able to facilitate the uptake in to cells and/or the intracellular release of an oligonucleotide from vesicles, e.g. endosomes or lysosomes. Therefore, in a preferred aspect, an exon 50 skipping AON according to the invention is formulated in a composition or a medicament or a composition, which is provided with at least an excipient and/or a targeting ligand for delivery and/or a delivery device thereof to a cell and/or enhancing its intracellular delivery.

It is to be understood that if a composition comprises an additional constituent such as an adjunct compound as defined herein, each constituent of the composition may not be formulated in one single combination or composition or preparation. Depending on their identity, the skilled person will know which type of formulation is the most appropriate for each constituent as defined herein. In a preferred embodiment, the invention provides a composition or a preparation which is in the form of a kit of parts comprising an exon 50 skipping AON according to the invention and a further adjunct compound as defined herein. If required, an exon 50 skipping AON according to the invention or a vector, preferably a viral vector, expressing an exon 50 skipping AON according to the invention can be incorporated into a pharmaceutically active mixture by adding a pharmaceutically acceptable carrier. Accordingly, the invention also provides a composition, preferably a pharmaceutical composition, comprising an exon 50 skipping AON according to the invention, or a viral vector according to the invention and a pharmaceutically acceptable excipient. Such composition may comprise a single exon 50 skipping AON or viral vector according to the invention, but may also comprise multiple, distinct exon 50 skipping AON or viral vectors according to the invention. Such a pharmaceutical composition may comprise any pharmaceutically acceptable excipient, including a carrier, filler, preservative, adjuvant, solubilizer and/or diluent. Such pharmaceutically acceptable carrier, filler, preservative, adjuvant, solubilizer and/or diluent may for instance be found in Remington (Remington. 2000. The Science and Practice of Pharmacy, 20th Edition. Baltimore, Md.: Lippincott Williams Wilkins). Each feature of said composition has earlier been defined herein.

A preferred route of administration is through direct intravitreal injection of an aqueous solution or specially adapted formulation for intraocular administration. EP2425814 discloses an oil in water emulsion especially adapted for intraocular (intravitreal) administration of peptide or nucleic acid drugs. This emulsion is less dense than the vitreous fluid, so that the emulsion floats on top of the vitreous, avoiding that the injected drug impairs vision.

If multiple distinct exon 50 skipping AONs according to the invention are used, concentration or dose defined herein may refer to the total concentration or dose of all AONs used or the concentration or dose of each exon 50 skipping AONs used or added. Therefore, in one embodiment, there is provided a composition wherein each or the total amount of exon 50 skipping AONs according to the invention used is dosed in an amount as disclosed herein.

A preferred USH2A exon 50 skipping AON according to the invention is for the treatment of an USH2A-related disease or condition of an individual. In all embodiments of the invention, the term ‘treatment’ is understood to include also the prevention and/or delay of the USH2A-related disease or condition. An individual, which may be treated using an exon 50 skipping AON according to the invention may already have been diagnosed as having a USH2A-related disease or condition. Alternatively, an individual which may be treated using an exon 50 skipping AON according to the invention may not have yet been diagnosed as having a USH2A-related disease or condition but may be an individual having an increased risk of developing a USH2A-related disease or condition in the future given his or her genetic background. A preferred individual is a human individual. In a preferred embodiment the USH2A-related disease or condition is Usher syndrome type II.

A treatment in a use or in a method according to the invention is at least once a week, once a one month, once every several months, once every 1, 2, 3, 4, 5, 6 years or longer, such as lifelong. Each exon 50 skipping AON or equivalent thereof as defined herein for use according to the invention may be suitable for direct administration to a cell, tissue and/or an organ in vivo of individuals already affected or at risk of developing USH2A-related disease or condition, and may be administered directly in vivo, ex vivo or in vitro. The frequency of administration of an AON, composition, compound or adjunct compound of the invention may depend on several parameters such as the severity of the disease, the age of the patient, the mutation of the patient, the number of exon 50 skipping AONs (i.e. dose), the formulation of said AON(s), the route of administration and so forth. The frequency may vary between daily, weekly, at least once in two weeks, or three weeks or four weeks or five weeks or a longer time period. Dose ranges of an exon 50 skipping AON according to the invention are preferably designed based on rising dose studies in clinical trials (in vivo use) for which rigorous protocol requirements exist. In a preferred embodiment, a viral vector, preferably an AAV vector as described earlier herein, as delivery vehicle for a molecule according to the invention, is administered in a dose ranging from 1×10⁹ to 1×10¹⁷ virus particles per injection, more preferably from 1×10¹⁰ to 1×10¹² virus particles per injection. The ranges of concentration or dose of AONs as given above are preferred concentrations or doses for in vivo, in vitro or ex vivo uses. The skilled person will understand that depending on the AONs used, the target cell to be treated, the gene target and its expression levels, the medium used and the transfection and incubation conditions, the concentration or dose of AONs used may further vary and may need to be optimized any further.

An exon 50 skipping AON according to the invention, or a viral vector according to the invention, or a composition according to the invention for use according to the invention may be suitable for administration to a cell, tissue and/or an organ in vivo of individuals already affected or at risk of developing a USH2A-related disease or condition, and may be administered in vivo, ex vivo or in vitro. As Usher syndrome type II has a pronounced phenotype in retina and inner ear cells, it is preferred that said cells are retina or inner ear cells, it is further preferred that said tissue is the retina or the inner ear and/or it is further preferred that said organ is the eye or the ear. Contacting the eye or ear cell with an exon 50 skipping AON according to the invention, or a viral vector according to the invention, or a composition according to the invention may be performed by any method known by the person skilled in the art. Use of the methods for delivery of exon 50 skipping AONs, viral vectors and compositions described herein is included. Unless otherwise indicated each embodiment as described herein may be combined with another embodiment as described herein.

The sequence information as provided herein should not be so narrowly construed as to require inclusion of erroneously identified bases. The skilled person can identify such erroneously identified bases and knows how to correct for such errors. All patent and literature references cited in the present specification are hereby incorporated by reference in their entirety.

### EXAMPLES

### Example 1. Providing and Testing Antisense Oligonucleotides (AONs) for Efficient Skipping of Exon 50 in Human USH2A Pre-mRNA

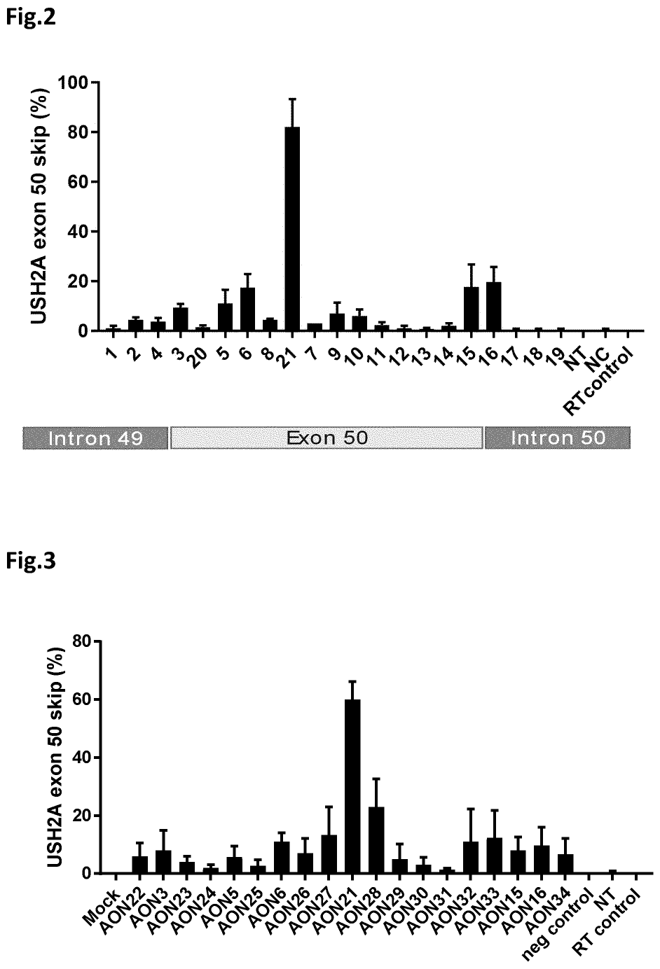

The sequence of exon 50 of the human USH2A gene and its surrounding intron sequences were analyzed for the presence of exonic splice enhancer (ESE) motifs. Multiple sites were initially determined (data not shown) and over time in total fifty AONs (AONs1-50) were manufactured in-house based on these ESE findings. Two of these AONs were known from the art: Radboud-1 (AON20) and Radboud-2 (AON21), see WO 2016/005514. However, when used apart, these AONs did not result in much exon 50 skipping, whereas when used together in a single administration, relatively good exon 50 skipping was observed (FIG. 4b in WO 2016/005514). The aim of the present study was to see whether new and more efficacious AONs could be generated to obtain significantly improved exon 50 skipping from human USH2A pre-mRNA, which may prove clinically relevant. Initially all AONs were modified with a 2′-O-methoxyethyl (2′-MOE) group at the sugar chain and all had a full phosphorothioated (PS) backbone. AONs were kept dissolved in water. The sequences of all fifty AONs are given in FIG. 1, under the target sequence of exon 50 of the human USH2A gene (this is given in FIG. 1 as DNA, but the target is pre-mRNA), and part of its surrounding intron sequences. The sequences of AON1 to AON50 are represented by SEQ ID NO:1 to 50. As becomes clear in FIG. 1, some AONs are partly complementary to an exon sequence at the 5′ end of exon 50, overlap the intron/exon boundary and are partly complementary to an intron 49 sequence upstream of exon 50 (AON4 and AON22), while other AONs are partly complementary to an exon sequence at the 3′ end of exon 50, overlap the exon/intron boundary and are partly complementary to an intron 50 sequence downstream of exon 50 (exemplified by AON44 to AON50). FIG. 1 also shows the sequence of Radboud-1 and Radboud-2 AONs (italic) which have their targeting sequence in the 5′ half of exon 50. In a first screen, AONs 1 to 19 were tested for exon 50 skipping efficiency and compared to AON20 (Radboud-1) and AON21 (Radboud-2). Negative controls were the following: no transfection (NT), an unrelated negative control (NC) AON (5′-CGU UCU CCA GGA AAG CCG AUG-3′; SEQ ID NO:107) and a Reverse Transcription control (RT). The following procedures were performed.

### Cell Culture and Transfection

The WERI-Rb1 (HTB-169TH) retinoblastoma cell line was obtained from ATCC. Cells were cultured in RPMI 1640 medium (Gibco) supplemented with 10% FBS. WERI-Rb1 is a suspension cell line and was maintained by addition of fresh medium or replacement of medium every 3 to 4 days. When passaging the cells, the concentration of the cells was kept at 3×10⁵ cells per mL, at 37° C. and 5% CO₂.

For transfection, cells were seeded at a concentration of 5×10⁵ cells in 3.8 cm² wells in 0.9 mL RPMI 1640 supplemented with 10% FBS in a 12-well plate. The next day cells were transfected with 50 nM oligonucleotides using Lipofectamine 2000 transfection reagent (Invitrogen). As a negative control, non-transfected (NT) and mock transfected cells were taken along. A ratio of 17 pmol AON to 1 μL Lipofectamine 2000 (5 μL of 10 μM AON stock and 3 μL Lipofectamine per well) was used. Both Lipofectamine 2000 and AON were prepared in Opti-MEM. Per condition, 50 μL of the AON mixture was added to 50 μL Lipofectamine 2000 mixture and incubated for 20 min at RT before adding the transfection complexes to the cells. Cells were incubated for 48 h at 37° C. Transfections were performed in triplicate.

RNA Isolation and cDNA Synthesis

Total RNA was isolated from the cells using the RNeasy Plus Mini Kit (Qiagen) according to the manufacturer's protocol. RNA was eluted in 50 μL RNase free water and the concentrations were measured on the Nanodrop 2000. Samples were stored at −80° C. cDNA was synthesized using 500 ng total RNA. A 20 μL reaction contained 1 μL Verso Reverse Transcriptase enzyme, 4 μL 5× cDNA buffer, 2 μL dNTP mix [5 mM], 1 μL RT enhancer and 1 μL Random Hexamer Primers [400 ng/μL] (Thermo Scientific). The reaction was run in a thermocycler for 30 min at 42° C., 2 minutes at 95° C. and kept at 4-12° C. Samples were stored at −20° C.

ddPCR Analysis

For the quantification of USH2A Δexon 50, ddPCR was performed with 100 ng WERI-Rb1 mRNA(/cDNA) using ddPCR supermix for probes (no dUTP) (Bio-Rad) in a multiplex manner. The final 21 μL reaction mix contained 1×Supermix, 0.6 μM USH2A Δexon 50 assay, and 0.6 μM of the USH2A Exon 62 reference assay. Primer and probe sequences were as follows:

USH2A Δexon 50 forward: 5′-GCTAAGGTTTGTTGTAACGG-3′ (SEQ ID NO:109)
USH2A Δexon 50 reverse: 5′-TTCACATAATCCTGCCCAC-3′ (SEQ ID NO:110)
Probe exon 50: 5′-/SFAM/CATACCTGG/ZEN/TAGAATTCTAGCGTAATACCC/3IABkFQ/-3′ (SEQ ID NO:111)
USH2A exon 62 ref forward: 5′-CGGGCATTGCTACTACAGTG-3′ (SEQ ID NO:112)
USH2A exon 62 ref reverse: 5′-CGGCGGAAGAGAAACTGAC-3′ (SEQ ID NO:113)
Probe exon 62: 5′-/56-HEX/CAGAGTACT/ZEN/CCAGGAACCCGTCACTGAAGA /3IABkFQ/-3′ (SEQ ID NO:114)

PCR reactions were dispersed into droplets using the QX200 droplet generator (Bio-Rad) according to the manufacturer's instructions and transferred to a 96-well PCR plate. End point PCR was performed in a T100 Thermocycler (Bio-Rad). The ddPCR protocol was as follows: The polymerase was heat activated at 95° C. for 10 minutes. Each cycle denaturation was performed at 94° C. for 30 seconds, annealing/extension at 60° C. for 1 minute in 40 cycles, enzyme deactivation at 98° C. for 10 min and kept indefinitely at 4° C. till further analysis. The fluorescence of each droplet was quantified in the QX200 droplet reader (Bio-Rad). Each sample was analyzed in duplicate. Absolute quantification was performed in QuantaSoft software (Bio-Rad). Thresholds were manually set to distinguish between positive and negative droplets (FAM at 4000, and HEX at 3000).

The primary analysis was performed using the QuantaSoft software. Only samples were included for further analysis when the total number of droplets was 10.000 per well. The negative control samples were checked for any application. The accepted samples were checked for both USH2A exon 62 reference values represented by the green (HEX) color and for USH2A exon 50 skipped values represented by the blue (FAM) droplets. Gating was performed manually separating the positive fluorescent cloud of droplets from the negative fluorescent droplets. After gating, the positive droplet counts in copies/20 μL for the two replicates was transported to an Excel file for secondary analysis. First the total copy numbers per sample for the two technical duplicates of each sample were averaged. Next the percentage skip was calculated by dividing the copies/20 μL found with the skip assay by those detected with the reference assay. Finally, the percentage skip per AON was calculated by averaging the two separate performed replicates and the standard error of mean (SEM) was derived from these final values.

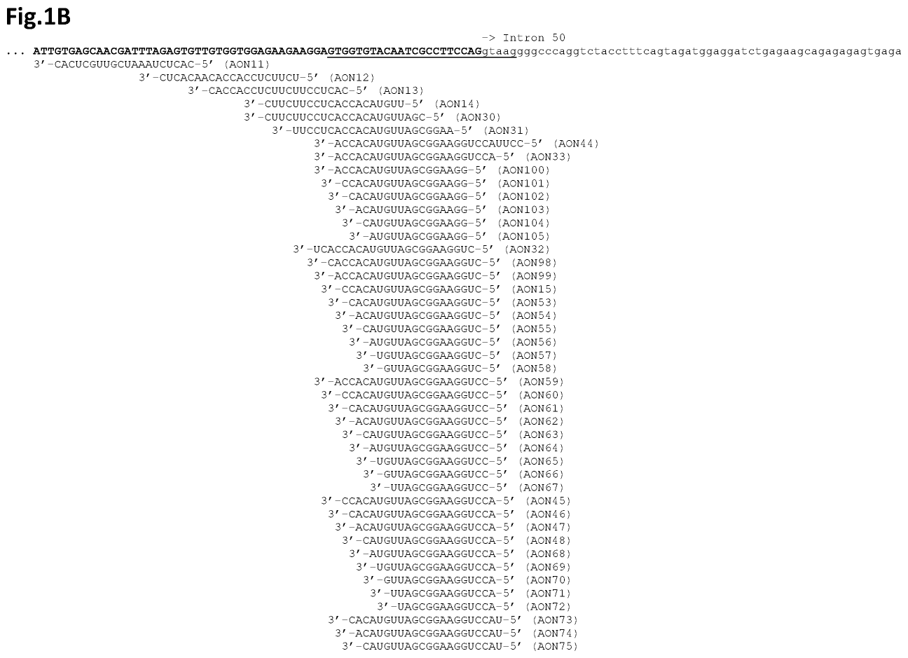

The results of the first screen are shown as percentage of exon 50 skip in the bar diagram of FIG. 2. The order of AONs on the x-axis was set to represent their distribution over the intron 49, exon 50 and intron 50 sequences given as bars below the diagram. FIG. 2 clearly shows that one of the prior art oligonucleotides, AON21 (Radboud-2) outperformed all 19 new AONs that were considered, whereas the other prior art oligonucleotide AON20 (Radboud-1) hardly gave any exon 50 skip. Notably, AON15 and AON16, targeting a sequence around the exon 50/intron 50 boundary gave a significant increased percentage of skip, in comparison to most other AONs tested, except then for AON21.

### Example 2. Testing Additional AONs for Efficient Skipping of Exon 50 in Human USH2A Pre-mRNA

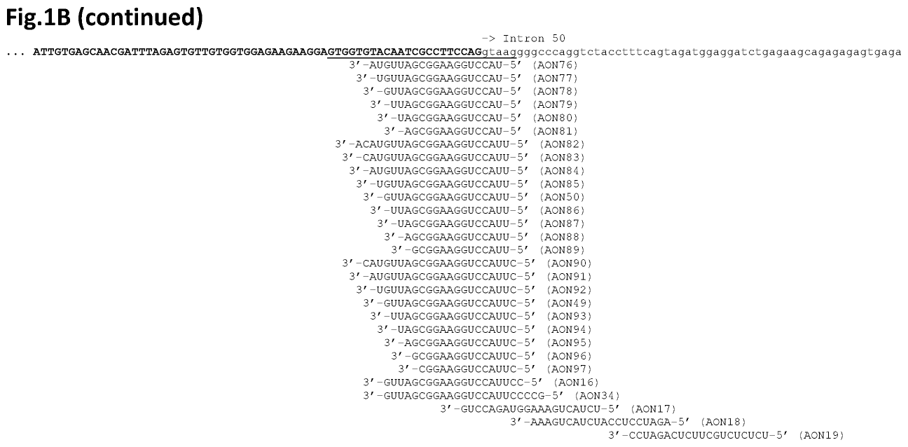

In a next experiment, more AONs that were targeting certain ‘hotspots’ for exon 50 skipping were tested in a transfection assay for their ability to provide exon 50 skipping. Transfection experiments were performed as outlined above. The following 2′-MOE modified oligonucleotides were tested: AON3, AON5, AON6, AON15, AON16, AON21, AON22, AON23, AON24, AON25, AON26, AON27, AON28, AON29, AON30, AON31, AON32, AON33, and AON34. Negative controls were mock, no transfection (NT), the negative control AON as used in the previous experiment and a reverse transcriptase control (RT). The results are shown in FIG. 3 that again has the AONs distributed such that they represent the targeting hotspots in exon 50 (AON22, -3, -23, -24, -5 and -25 targeting the first hotspot; AON6, -26, -27, -21, -28, and -29 targeting the second hotspot; and AON30, -31, -32, -33, -15, -16, and -34 targeting the third hotspot at the exon 50/intron 50 boundary). Even though the AONs that target the area of the 3′ terminus of exon 50 gave proper exon 50 skip, none of them was as efficient as AON21, using these procedures. The 23-mer AON28 (differing slightly from AON21) also showed efficient exon 50 skipping.

### Example 3. Testing Shorter AONs for Efficient Skipping of Exon 50 in Human USH2A Pre-mRNA

The inventors then questioned whether it would be beneficial and possible to reach higher exon 50 skipping percentages when shorter AONs were used. AON21, the oligonucleotide disclosed in WO 2016/005514 and shown to act quite efficiently (see above) is a 23-nucleotide long oligonucleotide. Possibly, shorter oligonucleotides would be able to enter cells more efficiently and eventually yield in higher exon 50 skip percentages in comparison to the relatively ‘long’ AON21. For this, the following oligonucleotides were tested for the hotspot surrounding the target sequence of AON21: AON21 (23 nt), AON27 (23 nt), AON28 (23 nt), AON35 (27 nt), AON36 (22 nt), AON37 (21 nt), AON38 (20 nt), AON39 (19 nt), AON40 (22 nt), AON41 (21 nt), AON42 (20 nt), AON43 (19 nt), AON16 (20 nt), AON33 (23 nt), AON44 (27 nt), AON45 (22 nt), AON46 (21 nt), AON47 (20 nt), AON48 (19 nt), AON49 (19 nt), and AON50 (18 nt). The results are given in FIG. 4. Even though many oligonucleotides (targeting the AON21 area, as well as those that target the 3′ terminus of exon 50) gave proper exon 50 skipping, reaching 30% or more, none of the oligonucleotides reached the percentage observed with AON21.

### Example 4. Testing Transfection Versus Gymnotic Uptake

Then, an experiment was performed that would be more representative of delivery of an oligonucleotide in a therapeutic setting. Whereas all experiments above were performed using transfection reagents, in a clinical administration, such transfection reagents are not applied and if one administers an oligonucleotide directly to the vitreous of the eye by naked intravitreal delivery, one relies on free uptake of the oligonucleotide by the target cells (in the retina, in the case of Usher syndrome) and trafficking through the cell towards and into the nucleus. It has been shown that such could be achieved in clinical trials using AONs for LCA10. However, for compound selection, as outlined above, often transfection experiments are performed on cells to reach high levels and distinguishable levels of exon skipping percentages. However, to determine which of the AONs would perform to what extent under more physiological conditions and so-called ‘gymnotic’ uptake (without transfection reagents) experiment was performed as follows: WERI-Rb1 cells were seeded at 5×10⁵ cells/well in a 12-wells plate. After an overnight incubation at 5% CO₂ and 37° C., AON was added to the cells to a final concentration of 10 μM in a total volume of 1 mL. The cells were subsequently cultured for 72 h, after which the cells were collected for RNA isolation. Initially AON21, AON35, AON36 and AON37 were used (targeting the area of AON21) and compared to AON46, AON47 and AON48 (targeting the 3′ terminal part of exon 50).

The results are shown in FIG. 5. This shows, much to the surprise of the inventors, that the percentage of exon 50 skip after application of AON21 was quite low, whereas the percentage observed with the three oligonucleotides that targeted the 3′ terminus of exon 50 gave—in contrast—very significant exon 50 skipping (FIG. 5B). These percentages were (not surprisingly) lower than observed with transfection (FIG. 5A), because transfection enormously boosts cell entry. In any case, using this gymnotic approach, AON48 was able to give approximately 10% exon 50 skipping, whereas AON21 gave approximately only 2%.

The reason for the dramatic difference in exon skipping percentage seen with AON21 after transfection and gymnotic uptake is likely due to secondary structures of the oligonucleotide. Such structures are no longer present after application of transfection reagents, while they remain under gymnotic circumstances. Hence, it is beneficial to not only shorten the oligonucleotide, but also to assess the potency of entering cells under more physiological conditions such as outlined herein, by indirectly assessing any secondary structures that may hamper cell entry (or skipping efficiency in the end). Surprisingly it was found that another targeting area than previously identified allowed significantly better exon 50 skipping results. It is concluded that the inventors of the present invention were able to identify a new area within the USH2A pre-mRNA that could be targeted to obtain a high percentage of exon 50 skipping. This area entails the ultimate 22 nucleotides of the 3′ terminus of exon 50 of human USH2A and the most 5′ located 5 nucleotides of intron 50 of human USH2A.

### Example 5. Testing Additional AONs after Gymnotic Uptake

Based on the results in the previous example, another set of AONs were tested in a gymnotic uptake experiment, again using WERI-RB1 cells using the same concentration of and conditions as outlined above. The results shown in FIG. 6 strikingly show that AON21 and AON43 that are located towards the 5′ end of exon 50 are outperformed by many AONs that target the 3′ area of exon 50 of human USH2A, with AON48, AON55, AON56 and AON103 performing best.

### Example 6. Exon 50 Skip in Human Organoids

A selection of AONs (AON3, AON21, AON43, AON48, AON55, AON101, and AON103) was tested on wildtype human eye-cup organoids (3D free-floating mini eyes, cultured in ultra-low adhesion 96-wells plates). The organoids were treated with two different concentrations (1.5 and 7.5 μM AON). A fully 2′-MOE and PS modified and unrelated negative control oligonucleotide at concentration 7.5 μM was taken along.

At day 0 the organoids were treated with 200 μL AON diluted in neural retina maintenance medium (NR medium: DMEM/F12 without HEPES; supplemented with 10% FBS, 1% N-2, 1× Glutamax, and 1% pen/strep). The organoids were incubated at 37° C., 5% CO₂ and 40% O₂. Every other day half of the medium with AON was removed and replaced with only NR medium (=wash out study). After four weeks the organoids were harvested and washed with 300 μL PBS in an Eppendorf tube. The PBS was completely removed and 300 μL TRIreagent (Sigma) was added to inhibit RNase activity. The organoids were snap frozen in liquid nitrogen and stored at −80° C.

To isolate the RNA the organoids were thawed and ruptured using a P200 pipet tip by pipetting up and down 10 times, and further ruptured through a 25-gauge needle (10 times). The lysate was mixed thoroughly with 300 μL ethanol 96% and loaded to a Zymo-Spin IC Column (Zymo research). The RNA was isolated using the Direct-zol RNA microPrep kit according to the manufacturers protocol and eluted in 20 μL DNAse/RNase free water. The RNA concentration was measured using the nanodrop 2000 (Thermo Scientific). From 150 ng RNA template, cDNA was synthesized using random hexamers (600 ng) from the Verso cDNA synthesis kit (Thermo Scientific) in a total volume of 30 μL. Reverse transcriptase enhancer (1.5 μL/reaction) was included to remove possible double stranded DNA contamination in the RNA sample. Synthesis was performed at 42° C. for 30 min and enzyme inactivation was performed at 95° C. for 2 min. cDNA was stored at −20° C. until further analysis in ddPCR, which was performed to measure the Ush2A exon 50 skip values and the CRX values. CRX is a homeobox transcription factor essential for photoreceptor differentiation and was used as a retinal progenitor marker. It is an early marker and after differentiation into a retinal organoid, in time CRX expression is decreasing. The value of 1000 CRX copies/ng RNA is used as a threshold value to characterize differentiation in the samples.

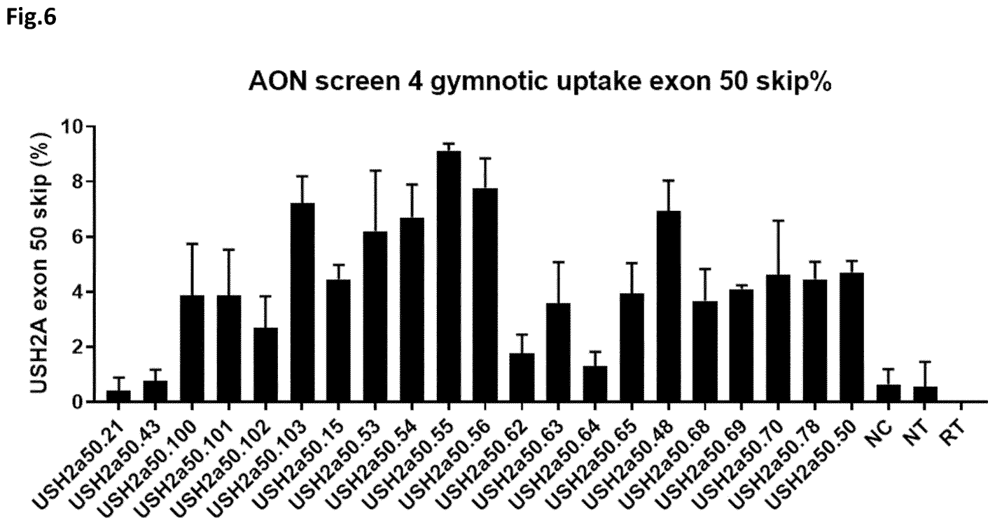

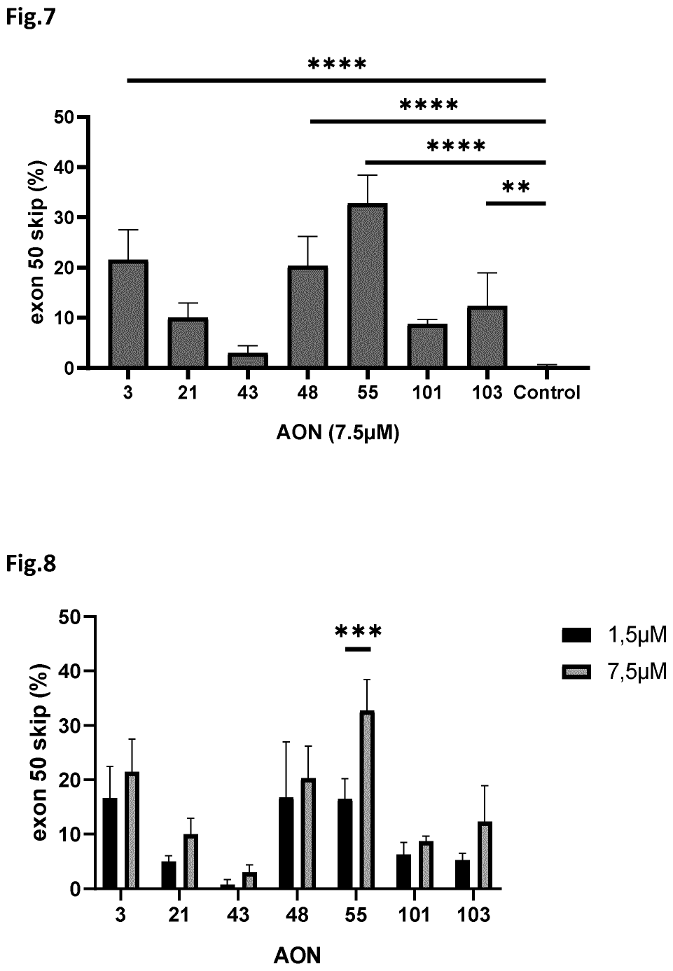

ddPCR sample was performed as in example 1. Each sample was measured in duplicate. Average values were used in the calculation. FIG. 7 shows that the organoids treated with AONs 3, 48, 55, and 103 experienced a significant increase in exon 50 skip in comparison to the control treated organoids. A dose dependent increase was observed with AON 55 (FIG. 8), which suggests that the low concentration of 1.5 μM was already sufficient to introduce maximum exon 50 skip levels, but this needs further investigation. These experiments in eye cups, that does not make use of transfections and allows the AONs to enter the cells (and even the correct tissue within the organoid) show that the inventors were able to identify several oligonucleotides (exemplified in this experiment by AON3, AON48 and AON55) that outperformed the oligonucleotide known from the art (AON21) and confirmed the data that was shown in the gymnotic uptake experiments of FIGS. 5B and 6, but also confirmed that short oligonucleotides (14 to 22 nt) and that target the sequence as represented by SEQ ID NO:53 can beneficially be used in the treatment of Usher syndrome type Ila, caused by mutations in exon 50 of the USH2A gene.

1. An antisense oligonucleotide (AON) capable of skipping exon 50 from human USH2A pre-mRNA, wherein the AON comprises a 14 to 22 consecutive nucleotide sequence that is 90% to 100% to a consecutive sequence within SEQ ID NO:53.

2. The AON according to claim 1, wherein the AON consists of 16, 17, 18, 19, 20, 21 or 22 nucleotides.

3. The AON according to claim 1, wherein the AON consists of a sequence selected from the group consisting of SEQ ID NO:48, 15, 16, 45, 46, 47, 49, 50, and 54 to 106, preferably SEQ ID NO:58, 59, 104, 48, 57, 56, 15, 73, 50, 81, 101, 102, 72, 68, 71 and 66.

4. The AON according to claim 1, wherein the AON is an oligoribonucleotide.

5. The AON according to claim 1, wherein the AON comprises at least one 2′-O-methoxyethyl (2′-MOE) modification.

6. The AON according to claim 5, wherein all nucleotides of the AON are 2′-MOE modified.

7. The AON according to claim 1, wherein the AON comprises at least one non-naturally occurring internucleoside.

8. The AON according to claim 7, wherein all sequential nucleosides are interconnected by phosphorothioate.

9. A viral vector expressing an AON according to claim 1.

10. A pharmaceutical composition comprising an AON according to claim 1 and a pharmaceutically acceptable carrier.

11. The AON according to claim 1, for use in the treatment, prevention or delay of an USH2A-related disease or a condition requiring modulating splicing of USH2A pre-mRNA.

12. The AON for use according to claim 11, wherein the AON is for intravitreal administration and is dosed in an amount ranging from 5 μg to 500 μg of total AON per eye.

13. The AON for use according to claim 12, wherein the AON is dosed in an amount ranging from 25 μg to 100 μg of total AON per eye.

14. Use of an AON according to claim 1, for the preparation of a medicament for the treatment, prevention or delay of an USH2A-related disease or a condition requiring modulating splicing of USH2A pre-mRNA.

15. An in vitro, ex vivo or in vivo method for modulating splicing of USH2A pre-mRNA in a cell, comprising the steps of: administering to the cell an AON according to claim 1; allowing the hybridization of the AON to its complementary sequence in USH2A target RNA molecule in the cell; and allowing the skip of exon 50 from the target RNA molecule.

16. A method for the treatment of a USH2A-related disease or condition requiring modulating splicing of USH2A pre-mRNA of an individual in need thereof, said method comprising contacting a cell of said individual with an AON according to claim 1.


SEQUENCE LISTING (114 sequences)

SEQ ID NO: 1 - DNA, 22 bp, Artificial sequence
  AON1
aucacaaucaucuugcagaucc

SEQ ID NO: 2 - DNA, 24 bp, Artificial sequence
  AON2
cugucaauuuaggacaauaauaau

SEQ ID NO: 3 - DNA, 20 bp, Artificial sequence
  AON3
aucuggacagcauacuucac

SEQ ID NO: 4 - DNA, 23 bp, Artificial sequence
  AON4
cagcauacuucaccugucaauuu

SEQ ID NO: 5 - DNA, 21 bp, Artificial sequence
  AON5
ccgauugugcuguucaucugg

SEQ ID NO: 6 - DNA, 20 bp, Artificial sequence
  AON6
aggaaucaccaaugccaaca

SEQ ID NO: 7 - DNA, 20 bp, Artificial sequence
  AON7
cugagguggaguacggcauu

SEQ ID NO: 8 - DNA, 20 bp, Artificial sequence
  AON8
cauucugccacagcaggaau

SEQ ID NO: 9 - DNA, 20 bp, Artificial sequence
  AON9
ccagcacagcaaaucugguu

SEQ ID NO: 10 - DNA, 20 bp, Artificial sequence
  AON10
ugucugccacagcacuucug

SEQ ID NO: 11 - DNA, 20 bp, Artificial sequence
  AON11
cacucuaaaucguugcucac

SEQ ID NO: 12 - DNA, 20 bp, Artificial sequence
  AON12
ucuucuccaccacaacacuc

SEQ ID NO: 13 - DNA, 20 bp, Artificial sequence
  AON13
cacuccuucuucuccaccac

SEQ ID NO: 14 - DNA, 20 bp, Artificial sequence
  AON14
uuguacaccacuccuucuuc

SEQ ID NO: 15 - DNA, 20 bp, Artificial sequence
  AON15
cuggaaggcgauuguacacc

SEQ ID NO: 16 - DNA, 20 bp, Artificial sequence
  AON16
ccuuaccuggaaggcgauug

SEQ ID NO: 17 - DNA, 20 bp, Artificial sequence
  AON17
ucuacugaaagguagaccug

SEQ ID NO: 18 - DNA, 20 bp, Artificial sequence
  AON18
agauccuccaucuacugaaa

SEQ ID NO: 19 - DNA, 20 bp, Artificial sequence
  AON19
ucucucugcuucucagaucc

SEQ ID NO: 20 - DNA, 24 bp, Artificial sequence
  Radboud-1 AON20
gugcuguucaucuggacagcauac

SEQ ID NO: 21 - DNA, 23 bp, Artificial sequence
  Radboud-2 AON21
aguacggcauucugccacagcag

SEQ ID NO: 22 - DNA, 23 bp, Artificial sequence
  AON22
caucuggacagcauacuucaccu

SEQ ID NO: 23 - DNA, 23 bp, Artificial sequence
  AON23
uguucaucuggacagcauacuuc

SEQ ID NO: 24 - DNA, 23 bp, Artificial sequence
  AON24
uugugcuguucaucuggacagca

SEQ ID NO: 25 - DNA, 23 bp, Artificial sequence
  AON25
gccaacagaaacccgauugugcu

SEQ ID NO: 26 - DNA, 23 bp, Artificial sequence
  AON26
uucugccacagcaggaaucacca

SEQ ID NO: 27 - DNA, 23 bp, Artificial sequence
  AON27
cggcauucugccacagcaggaau

SEQ ID NO: 28 - DNA, 23 bp, Artificial sequence
  AON28
guggaguacggcauucugccaca

SEQ ID NO: 29 - DNA, 23 bp, Artificial sequence
  AON29
uuccugagguggaguacggcauu

SEQ ID NO: 30 - DNA, 23 bp, Artificial sequence
  AON30
cgauuguacaccacuccuucuuc

SEQ ID NO: 31 - DNA, 23 bp, Artificial sequence
  AON31
aaggcgauuguacaccacuccuu

SEQ ID NO: 32 - DNA, 23 bp, Artificial sequence
  AON32
cuggaaggcgauuguacaccacu

SEQ ID NO: 33 - DNA, 23 bp, Artificial sequence
  AON33
accuggaaggcgauuguacacca

SEQ ID NO: 34 - DNA, 23 bp, Artificial sequence
  AON34
gccccuuaccuggaaggcgauug

SEQ ID NO: 35 - DNA, 27 bp, Artificial sequence
  AON35
guggaguacggcauucugccacagcag

SEQ ID NO: 36 - DNA, 22 bp, Artificial sequence
  AON36
aguacggcauucugccacagca

SEQ ID NO: 37 - DNA, 21 bp, Artificial sequence
  AON37
aguacggcauucugccacagc

SEQ ID NO: 38 - DNA, 20 bp, Artificial sequence
  AON38
aguacggcauucugccacag

SEQ ID NO: 39 - DNA, 19 bp, Artificial sequence
  AON39
aguacggcauucugccaca

SEQ ID NO: 40 - DNA, 22 bp, Artificial sequence
  AON40
guacggcauucugccacagcag

SEQ ID NO: 41 - DNA, 21 bp, Artificial sequence
  AON41
uacggcauucugccacagcag

SEQ ID NO: 42 - DNA, 20 bp, Artificial sequence
  AON42
acggcauucugccacagcag

SEQ ID NO: 43 - DNA, 19 bp, Artificial sequence
  AON43
uacggcauucugccacagc

SEQ ID NO: 44 - DNA, 27 bp, Artificial sequence
  AON44
ccuuaccuggaaggcgauuguacacca

SEQ ID NO: 45 - DNA, 22 bp, Artificial sequence
  AON45
accuggaaggcgauuguacacc

SEQ ID NO: 46 - DNA, 21 bp, Artificial sequence
  AON46
accuggaaggcgauuguacac

SEQ ID NO: 47 - DNA, 20 bp, Artificial sequence
  AON47
accuggaaggcgauuguaca

SEQ ID NO: 48 - DNA, 19 bp, Artificial sequence
  AON48
accuggaaggcgauuguac

SEQ ID NO: 49 - DNA, 19 bp, Artificial sequence
  AON49
cuuaccuggaaggcgauug

SEQ ID NO: 50 - DNA, 18 bp, Artificial sequence
  AON50
uuaccuggaaggcgauug

SEQ ID NO: 51 - DNA, 379 bp, Homo sapiens
atcagttgggatctgcaagatgattgtgataaaagttaattgttagtcatagcatatttttttaactgatgtaatgattattattgtcctaaattgacaggtgaagtatgctgtccagatgaacagcacaatcgggtttctgttggcattggtgattcctgctgtggcagaatgccgtactccacctcaggaaaccagatttgctgtgctgggaggcttcatgatggccatggccagaagtgctgtggcagacagattgtgagcaacgatttagagtgttgtggtggagaagaaggagtggtgtacaatcgccttccaggtaaggggcccaggtctacctttcagtagatggaggatctgagaagcagagagagtgaga

SEQ ID NO: 52 - DNA, 219 bp, Homo sapiens
gtgaagtatgctgtccagatgaacagcacaatcgggtttctgttggcattggtgattcctgctgtggcagaatgccgtactccacctcaggaaaccagatttgctgtgctgggaggcttcatgatggccatggccagaagtgctgtggcagacagattgtgagcaacgatttagagtgttgtggtggagaagaaggagtggtgtacaatcgccttccag

SEQ ID NO: 53 - DNA, 27 bp, Homo sapiens
gtggtgtacaatcgccttccaggtaag

SEQ ID NO: 54 - RNA, 22 nt, Artificial sequence
  AON98
cuggaaggcgauuguacaccac

SEQ ID NO: 55 - RNA, 21 nt, Artificial sequence
  AON99
cuggaaggcgauuguacacca

SEQ ID NO: 56 - RNA, 19 nt, Artificial sequence
  AON53
cuggaaggcgauuguacac

SEQ ID NO: 57 - RNA, 18 nt, Artificial sequence
  AON54
cuggaaggcgauuguaca

SEQ ID NO: 58 - RNA, 17 nt, Artificial sequence
  AON55
cuggaaggcgauuguac

SEQ ID NO: 59 - RNA, 16 nt, Artificial sequence
  AON56
cuggaaggcgauugua

SEQ ID NO: 60 - RNA, 15 nt, Artificial sequence
  AON57
cuggaaggcgauugu

SEQ ID NO: 61 - RNA, 14 nt, Artificial sequence
  AON58
cuggaaggcgauug

SEQ ID NO: 62 - RNA, 22 nt, Artificial sequence
  AON59
ccuggaaggcgauuguacacca

SEQ ID NO: 63 - RNA, 21 nt, Artificial sequence
  AON60
ccuggaaggcgauuguacacc

SEQ ID NO: 64 - RNA, 20 nt, Artificial sequence
  AON61
ccuggaaggcgauuguacac

SEQ ID NO: 65 - RNA, 19 nt, Artificial sequence
  AON62
ccuggaaggcgauuguaca

SEQ ID NO: 66 - RNA, 18 nt, Artificial sequence
  AON63
ccuggaaggcgauuguac

SEQ ID NO: 67 - RNA, 17 nt, Artificial sequence
  AON64
ccuggaaggcgauugua

SEQ ID NO: 68 - RNA, 16 nt, Artificial sequence
  AON65
ccuggaaggcgauugu

SEQ ID NO: 69 - RNA, 15 nt, Artificial sequence
  AON66
ccuggaaggcgauug

SEQ ID NO: 70 - RNA, 14 nt, Artificial sequence
  AON67
ccuggaaggcgauu

SEQ ID NO: 71 - RNA, 18 nt, Artificial sequence
  AON68
accuggaaggcgauugua

SEQ ID NO: 72 - RNA, 17 nt, Artificial sequence
  AON69
accuggaaggcgauugu

SEQ ID NO: 73 - RNA, 16 nt, Artificial sequence
  AON70
accuggaaggcgauug

SEQ ID NO: 74 - RNA, 15 nt, Artificial sequence
  AON71
accuggaaggcgauu

SEQ ID NO: 75 - RNA, 14 nt, Artificial sequence
  AON72
accuggaaggcgau

SEQ ID NO: 76 - RNA, 22 nt, Artificial sequence
  AON73
uaccuggaaggcgauuguacac

SEQ ID NO: 77 - RNA, 21 nt, Artificial sequence
  AON74
uaccuggaaggcgauuguaca

SEQ ID NO: 78 - RNA, 20 nt, Artificial sequence
  AON75
uaccuggaaggcgauuguac

SEQ ID NO: 79 - RNA, 19 nt, Artificial sequence
  AON76
uaccuggaaggcgauugua

SEQ ID NO: 80 - RNA, 18 nt, Artificial sequence
  AON77
uaccuggaaggcgauugu

SEQ ID NO: 81 - RNA, 17 nt, Artificial sequence
  AON78
uaccuggaaggcgauug

SEQ ID NO: 82 - RNA, 16 nt, Artificial sequence
  AON79
uaccuggaaggcgauu

SEQ ID NO: 83 - RNA, 15 nt, Artificial sequence
  AON80
uaccuggaaggcgau

SEQ ID NO: 84 - RNA, 14 nt, Artificial sequence
  AON81
uaccuggaaggcga

SEQ ID NO: 85 - RNA, 22 nt, Artificial sequence
  AON82
uuaccuggaaggcgauuguaca

SEQ ID NO: 86 - RNA, 21 nt, Artificial sequence
  AON83
uuaccuggaaggcgauuguac

SEQ ID NO: 87 - RNA, 20 nt, Artificial sequence
  AON84
uuaccuggaaggcgauugua

SEQ ID NO: 88 - RNA, 19 nt, Artificial sequence
  AON85
uuaccuggaaggcgauugu

SEQ ID NO: 89 - RNA, 17 nt, Artificial sequence
  AON86
uuaccuggaaggcgauu

SEQ ID NO: 90 - RNA, 16 nt, Artificial sequence
  AON87
uuaccuggaaggcgau

SEQ ID NO: 91 - RNA, 15 nt, Artificial sequence
  AON88
uuaccuggaaggcga

SEQ ID NO: 92 - RNA, 14 nt, Artificial sequence
  AON89
uuaccuggaaggcg

SEQ ID NO: 93 - RNA, 22 nt, Artificial sequence
  AON90
cuuaccuggaaggcgauuguac

SEQ ID NO: 94 - RNA, 21 nt, Artificial sequence
  AON91
cuuaccuggaaggcgauugua

SEQ ID NO: 95 - RNA, 20 nt, Artificial sequence
  AON92
cuuaccuggaaggcgauugu

SEQ ID NO: 96 - RNA, 18 nt, Artificial sequence
  AON93
cuuaccuggaaggcgauu

SEQ ID NO: 97 - RNA, 17 nt, Artificial sequence
  AON94
cuuaccuggaaggcgau

SEQ ID NO: 98 - RNA, 16 nt, Artificial sequence
  AON95
cuuaccuggaaggcga

SEQ ID NO: 99 - RNA, 15 nt, Artificial sequence
  AON96
cuuaccuggaaggcg

SEQ ID NO: 100 - RNA, 14 nt, Artificial sequence
  AON97
cuuaccuggaaggc

SEQ ID NO: 101 - RNA, 19 nt, Artificial sequence
  AON100
ggaaggcgauuguacacca

SEQ ID NO: 102 - RNA, 18 nt, Artificial sequence
  AON101
ggaaggcgauuguacacc

SEQ ID NO: 103 - RNA, 17 nt, Artificial sequence
  AON102
ggaaggcgauuguacac

SEQ ID NO: 104 - RNA, 16 nt, Artificial sequence
  AON103
ggaaggcgauuguaca

SEQ ID NO: 105 - RNA, 15 nt, Artificial sequence
  AON104
ggaaggcgauuguac

SEQ ID NO: 106 - RNA, 14 nt, Artificial sequence
  AON105
ggaaggcgauugua

SEQ ID NO: 107 - RNA, 21 nt, Artificial sequence
  Control AON
cguucuccaggaaagccgaug

SEQ ID NO: 108 - RNA, 19 nt, Artificial sequence
  AON48*
acauggaaggcgauuguac

SEQ ID NO: 109 - DNA, 20 bp, Artificial sequence
  USH2A delta exon 50 forward primer
gctaaggtttgttgtaacgg

SEQ ID NO: 110 - DNA, 19 bp, Artificial sequence
  USH2A delta exon 50 reverse primer
ttcacataatcctgcccac

SEQ ID NO: 111 - DNA, 30 bp, Artificial sequence
  Probe exon 50
catacctggtagaattctagcgtaataccc

SEQ ID NO: 112 - DNA, 20 bp, Artificial sequence
  USH2A exon 62 reference forward primer
cgggcattgctactacagtg

SEQ ID NO: 113 - DNA, 19 bp, Artificial sequence
  USH2A exon 62 reference reverse primer
cggcggaagagaaactgac

SEQ ID NO: 114 - DNA, 30 bp, Artificial sequence
  Probe exon 62
cagagtactccaggaacccgtcactgaaga